(19) United States
(12) Patent Application Publication
(10) Pub. No.: US 2017/0173213 A1
(43) Pub. Date: Jun. 22, 2017

(54) POLARIZED HYDROXYAPATITE FILMS AND METHODS OF MAKING AND USING SAME

(71) Applicant: University of Rochester, Rochester, NY (US)

(72) Inventors: Matthew YATES, Fairport, NY (US); Keith SAVINO, Rochester, NY (US); Paul GABRYS, Eagle River, AR (US); Cong FU, Rochester, NY (US)

(21) Appl. No.: 15/124,401

(22) PCT Filed: Mar. 10, 2015
(86) PCT No.: PCT/US15/19601

§ 371 Date: Sep. 8, 2016

Related U.S. Application Data
(60) Provisional application No. 61/950,479, filed on Mar. 10, 2014.

(51) Int. Cl. A61L 27/32; A61L 31/08; A61K 33/42; C25D 9/04; A61L 31/16; A61K 33/38; C25D 3/46; A61L 27/30; A61L 27/54

(52) U.S. Cl. CPC A61L 27/32; A61L 27/306; A61L 31/086; A61L 31/088; A61K 33/42; A61L 27/54; A61L 31/16; A61K 33/38; C25D 3/46; C25D 9/04; A61L 2400/12; A61L 2400/18; A61L 2420/02; A61L 2430/02; A61L 2300/404

(57) ABSTRACT

Polarized hydroxyapatite films disposed on a substrate. The films have a residual polarization of at least 5 mC/cm2. Also provided are methods of making and using polarized hydroxyapatite. The films can be used as coatings of medical devices, such as, for example, medical implants.

### CROSS-REFERENCE TO RELATED APPLICATIONS

This application claims priority to U.S. Provisional Application No. 61/950,479, filed on Mar. 10, 2014, now pending, the disclosure of which is incorporated herein by reference.

### STATEMENT REGARDING FEDERALLY SPONSORED RESEARCH

This invention was made with government support under Grant Nos. 0856128 and 1343083 awarded by the National Science Foundation. The government has certain rights in the invention.

### BACKGROUND OF THE DISCLOSURE

Hydroxyapatite (HA) is a crystalline calcium phosphate that is the primary mineral component of teeth and bone, and the osteoconductive properties of synthetic HA has led to its widespread use as a coating or additive in bone grafts, scaffolds, and orthopedic implants. The HA crystals can exist in either monoclinic or hexagonal symmetry with each type having columns of hydroxyl ions along the crystallographic c-axis. The hydroxyl ion columns give rise to a number of observed electrical properties of the crystals, including high temperature proton conductivity, ferroelectricity, and electret behavior.

The orthopedic implant market in 2012 was valued at just over $30.5 billion, with over 2.6 million orthopedic implants inserted annually in the United States alone. Implants are typically composed of titanium or titanium alloys due to its biocompatability, but much work has been done to enhance the integration process by coating implant surfaces with hydroxyapatite (HA, Ca₅(PO₄)₃OH). HA's similar composition to bone provides a bioactive surface to form better tissue ingrowth between the patient's body and the orthopedic implant, helping to speed up the recovery process and prevent future prosthetic loosening. However, titanium or hydroxyapatite coated implants do very little in regards to preventing infections. Over 100,000 implants are infected each year in the United States alone, with medical costs exceeding $3 billion to treat these infections. Even in the most sterile surgical environments, bacteria from the surgical equipment, clothing from medical staff, or a patient's own skin can still adhere to an implant. Infected implants can be devastating to a patient and may even require a secondary surgery to clean or replace the implant. In worst case scenarios, infected implants can lead to amputation or even lethal sepsis. Therefore, there is an urgency to not just treat infections, but prevent them from occurring in the first place. If bacteria adhesion occurs and a bacteria biofilm forms on an implant before tissue regeneration occurs, the biofilm can be ten to one thousand times more resistant to antimicrobial agents than free-floating bacteria. While systemic antibiotic delivery can help prevent infections, the large dosage of drugs used increases the likelihood of a patient suffering negative side effects. Also, continual administration of antibiotics may cause antibiotic-resistant bacteria to form. Therefore, much research has been done to apply antibiotics locally to the surgical site. Not only can local drug delivery reduce the amount of antibiotic administered, it is also more likely to prevent an infection due to its close proximity to the surgical site.

To locally administer drugs, many surgeons have used bone cements made of polymethylmethacrylate (PMMA) that are loaded with antibiotics. However, PMMA does nothing to help stimulate osteointegration of the implant with the body and may even need to be removed in a subsequent surgery. Many groups have also tried to load drugs into HA coated titanium implants. However, the amount of antibiotic loaded and the release profile usually relies on a bulk diffusion mechanism or surface roughness characteristics, limiting the parameters to enhance the amount of loading and extend the release time of antibiotics, particularly for smaller antibiotics. Electrostatically charged hydrogels have been synthesized in an effort to extend the drug release, but they have very little benefit in terms of helping the implant integrate into the body.

### BRIEF DESCRIPTION OF THE FIGURES

FIG. 1. Example of electrochemically deposited HA crystals on a titanium substrate. The rod shaped crystals are visible on the upper right of the image. The lower left of the image is the underlying titanium where the crystals have been scraped away.

FIGS. 2A-2B. Example of a dense carbonated HA coating on titanium following four repeated hydrothermal growth steps at 200° C. for 10 hours each. FIG. 2A is the top view image and FIG. 2B is the side view image of a section that has been scraped away.

FIGS. 3A-3B. TSDC measurement of an example of a dense carbonated HA sample (FIG. 3A) and an electrochemically deposited HA (FIG. 3B).

FIGS. 4A-4B. Example of HA coatings after immersion in 1.5 times simulated body fluid (SBF) solutions for 24 h. FIG. 4A shows an as synthesized coating.

FIG. 4B shows a coating that was depolarized by heating to 600° C. prior to submersing in SBF. The rod shaped HA crystals are visible at the base of the coating. The newly deposited HA appears as a porous layer at the top of the coating.

FIG. 5. X-ray diffraction pattern from an example of a hydroxyapatite seed layer electrochemically synthesized on titanium. Peaks due to hydroxyapatite are identified with their corresponding Miller index number. Peaks due to reflections from the underlying titanium substrate are identified with the + symbol. Enhancement of the (002) peak intensity relative to other peaks indicates preferential crystal orientation with the c-axis normal to the titanium substrate.

FIG. 6. X-ray diffraction pattern of an example of carbonated hydroxyapatite grown hydrothermally onto the electrochemically synthesized seed layer. Strong enhancement of the intensity of the (002) and (004) peaks indicates crystal orientation with the c-axis normal to the titanium substrate.

FIG. 7. FTIR spectra of an example of a carbonated HA sample. The absorption bands at 958 cm⁻¹ and 1006 cm⁻¹ are ascribed to the PO₄³⁻ group. Absorption of the CO₃²⁻ group at 872, 1407, and 1452 cm⁻¹ was observed. The CO₃²⁻ absorption bands indicate the as-synthesized coating is B-type carbonated hydroxyapatite (CHA). Therefore, the stoichiometric formula Ca₁₀₋ₓ(PO₄)₆₋ₓ(CO₃)ₓ(OH)₂₋ₓ can be applied. With the help of elemental analysis, the carbon content was determined as 1.22%, giving x=1 in the stoichiometric formula.

FIG. 8. TSDC measurement repeated on three separate examples of carbonated HA samples, labeled (i), (ii), and (iii). Each sample was heated at a rate of 5° C. per minute as current was measured. All three samples show similar large peak current densities of ˜130 μA/cm² when the sample reached ˜430° C. The average calculated stored charge from TSDC data of the three samples is 73 mC/cm².

FIGS. 9A-9D. XRD patterns and SEM images of control samples. FIGS. 9A and 9B are for HA prepared by plasma spray onto titanium. FIGS. 9C and 9D are for carbonated HA synthesized hydrothermally on a titanium substrate seeded without electrochemical synthesis. The XRD patterns are consistent with the HA crystal structure. The carbonated HA has strong preferential orientation of the c-axis normal to the substrate, as evident by the coating morphology in the SEM image (FIG. 9D) and the enhancement of (002) and (004) peaks in the XRD pattern (FIG. 9C).

FIG. 10. TSDC data for an example of carbonated HA grown hydrothermally onto an electrochemically seeded titanium substrate (i) in comparison to carbonated HA grown hydrothermally onto a non-electrochemically seeded titanium substrate (ii), and HA deposited by plasma spray onto titanium (iii). The measured stored charge obtained from integration of the TSDC curves is 70 mC/cm² for (I), 0.5 mC/cm² for (ii), and ˜0 for (iii). The two small peaks for (ii) are consistent with proton migration and space charge polarization during dehydroxylation. It is considered that asymmetric dehydroxylation at the surface, and the resulting surface conversion of HA to beta-tricalcium phosphate upon dehydroxylation is responsible for the stored charge of the control sample (ii). The very large current density of (i) is due to the dipole formation in HA during electrochemical synthesis of the seed crystals.

FIGS. 11A-11C. XPS spectra of O 1s (FIG. 11A), Ca 2p3/2 (FIG. 11B) and P 2p (FIG. 11C) peaks from example HA seed layers with different electrochemical reaction times.

FIG. 12A-12D. Immunofluorescent staining of MSCs for vinculin (green), F-actin (red), and nuclear To-Pro3 (blue) on Ti (FIG. 12A), an example of a seed layer (FIG. 12B), an example of a heated seed layer at 300° C. (FIG. 12C), and an example of heated seed layer at 600° C. (FIG. 12D).

FIG. 13. Intersurface comparison of cell (nuclear) density showed that an untreated seed layer has a higher potential of cell viability than when heated at 300° C. or 600° C. to depolarize sample.

FIGS. 14A-14B. Example of HA nanocrystals electrochemically deposited on titanium (FIG. 14B). Example of dense carbonated HA coating grown hydrothermally onto the electrochemically seeded surface shown in FIG. 14A.

FIG. 15. Thermally stimulated depolarization current for an example of a carbonated HA coating on titanium.

FIGS. 16A-16C. Illustration of electric dipole formation in an example of electrochemically grown HA. Negative surface charge on Ti cathode (FIG. 16A). Calcium-rich HA nucleating on the TI surface (FIG. 16B). Concentration of positive calcium ions relative to negative phosphate and hydroxyl ions decreases with distance from the Ti surface, resulting in a permanent dipole moment illustrated by the arrow (FIG. 16C).

FIGS. 17A-17F. Example of new HA grown from simulated body fluid on a seed layer (FIG. 17A and FIG. 17B), and dense carbonated HA (FIG. 17C and FIG. 17D). Images in FIG. 17E and FIG. 17F are of the bottom of the dense sample before and after, respectively, exposure to simulated body fluid.

FIGS. 18A-18E. SEM of an example of HA side view (FIG. 18A), the example of HA top view (FIG. 18B), an example of AgHA side view (FIG. 18C), the example of AgHA top view (FIG. 18D), and an example of AgHA zoomed in (FIG. 18E).

FIG. 19. EDX spectrum of an example of an AgHA.

FIG. 20. XRD of an example of an HA and an example of an AgHA.

FIGS. 21A-21D. SEM of new HA growth after being immersed in SBF for 24 hours. FIG. 21A shows an example of HA after SBF immersion, FIG. 21B shows an example of AgHA after SBF immersion, FIG. 21C is a representative top view of samples in FIGS. 21A and 21B images, and FIG. 21D shows the Ti after SBF immersion.

FIG. 22. Growth of S. aureus bacteria when exposed to an example of HA and an example of AgHA.

FIG. 23. Silver nanoparticles appear on the tips of an example of rod-shaped HA crystals coating an underlying titanium substrate.

FIG. 24. Energy dispersive X-ray spectroscopy confirming silver deposition on the sample shown in FIG. 23.

FIG. 25. Silver nanoparticles along the entire exposed length of an example of HA crystals.

FIG. 26. A film of silver particles deposited over an example of HA crystals.

FIG. 27. Silver nanoparticles deposited onto the surface of an example of HA crystals synthesized via an electrochemical-hydrothermal synthesis.

### DETAILED DESCRIPTION OF THE DISCLOSURE

The present disclosure provides electrically polarized hydroxyapatite crystals and films of such crystals on a substrate. Also provided are methods of making and using the electrically polarized hydroxyapatite crystals and films.

It was surprisingly found that hydroxyapatite films comprised of HA crystals (e.g., rod-shaped crystals having a maximum dimensions of <1 micron (which can be referred to as seed crystals)) exhibit a desirable level of polarization (i.e., a polarization of at least at least 5 mC/cm²). The crystals are synthesized electrochemically from aqueous solution at relatively low temperature (<100° C.), short time (<5 minutes), and with a relatively low electric potential applied (<5 Volts).

In an aspect, the present disclosure provides electrically polarized hydroxyapatite crystals. In an embodiment, a composition comprises a plurality of electrically polarized hydroxyapatite crystals. In an embodiment, the electrically polarized hydroxyapatite crystals and films of such crystals are made by a method disclosed herein.

In an embodiment, a film comprises electrically polarized hydroxyapatite crystals. The films have a plurality of oriented crystals. The films have a stored charge such that they exhibit residual polarization. For example, the films have a stored charge of at least 5 mC/cm². The films are disposed on a substrate. The films may be comprised of only small seed crystals formed by electrochemical deposition methods. The films may be comprised of small seed crystals and crystals formed by hydrothermal methods.

For example, the films are synthesized from aqueous solution onto a substrate, and polarized during synthesis such that the HA film retains a positive electrostatic charge near the HA/substrate interface and a negative electrostatic charge near the HA/aqueous solution interface. The film is comprised of a plurality of HA crystals substantially oriented with the crystallographic c-axis normal to the substrate. The films may be comprised of HA crystals formed by electrochemical synthesis methods or a combination of electrochemical and chemical synthesis methods. The polarized crystals are retained on the substrate as a thin film, and may be optionally removed from the substrate by mechanical or chemical methods.

In an embodiment, film is a polarized hydroxyapatite film disposed on a substrate, the film comprising a plurality of HA crystals (e.g., rod shaped HA crystals) having a crystallographic c-axis substantially normal to the substrate, the film having a residual electrostatic charge of at least 5 mC/cm².

The film has HA crystals having a range of orientation relative to the substrate. A plurality of the crystals has a crystallographic c-axis substantially normal to the substrate. By substantially normal it is meant that at least 80% of the plurality of HA crystals have a crystallographic c-axis that deviates 20 degrees or less from normal to the substrate. In various embodiments, at least 80% to at least 99%, including all integer % values and ranges therebetween, of the plurality of HA crystals have a crystallographic c-axis that deviates 10 degrees or less to 20 degrees or less, including all integer degree values and ranges therebetween, from normal to the substrate. In an embodiment, at least 90% of the plurality of HA crystals have a crystallographic c-axis that deviates 20 degrees or less from normal to the substrate. In other embodiments, 100% of the plurality of HA crystals have a crystallographic c-axis that deviates 10, 9, 8, 7, 6, 5, 4, 3, 2, 1 degrees or less from normal to the substrate. The angle (degree) deviation of the crystallographic c-axis of the HA crystals can be determined by methods known in the art. For example, the angle (degree) deviation can be measured using an SEM image of a HA film or XRD pole figure analysis.

The film may have a gradient composition. Without intending to be bound by any particular theory, it is considered that the gradient composition of charged species (e.g., ions) is induced during electrochemical synthesis and that the gradient composition results in electrical polarization of the film. The gradient composition of the film can be determined by known analytical techniques. For example, the gradient composition of the film is determined by X-ray photoelectron microscopy.

In an embodiment, the polarized hydroxyapatite film, which may be synthesized on a substrate, comprises a plurality of hydroxyapatite crystals that have a positive charge near the substrate/hydroxyapatite interface, and a negative charge near the hydroxyapatite surface, resulting in the hydroxyapatite film having a residual electrostatic charge of at least 5 mC/cm².

The films have a residual electrostatic charge of at least 5 mC/cm². In various embodiments, the films have a residual electrostatic charge of at least 10 mC/cm², at least 15 mC/cm², or at least 20 mC/cm². In an embodiment, the film has a residual electrostatic charge of 5 mC/cm² to 100 mC/cm², including all integer mC/cm² values and ranges therebetween.

The film can have a range of thicknesses. For example, the film has a thickness of 100 nm to 50 microns, including all integer nm values and ranges therebetween. in an embodiment, the film has a thickness of 10 nm to 5000 microns, including all integer nm values and ranges therebetween.

A variety of substrates can be used. The substrate may be electrically conducting or non-conducting. Examples of suitable substrate materials include metals (e.g., titanium, copper, and platinum) and metal alloys (e.g., titanium/aluminum/vanadium, palladium/silver, and various grades of stainless steel), and other conducting materials such as graphite.

The substrate can have a variety of shapes. For example, if the substrate is planar, the direction of the crystallographic c-axis of the crystals is substantially normal to the substrate and if the substrate is curved, the direction of the crystallographic c-axis of the crystals follows the substrate curvature and is substantially normal to the substrate.

In an embodiment, the substrate is a medical implant or a portion thereof. The medical implant can be, for example, orthopedic implants, which may be used to support a damaged bone or to replace a missing joint or bone. Medical implants (e.g., orthopedic implants) can be used with or to replace or at least partially replace various parts of the skeletal system, including, for example, a hip, spine, neck, elbow, femur, knee, shoulder, arm, or finger. The implants, which may be fabricated of metal, may include pins, screws, plates, prostheses, nails, rods, or other devices. For example, the medical implant is an orthopedic implant such as an artificial hip (e.g., socket and/or ball), artificial spine, artificial neck, artificial elbow, artificial femur, artificial knee (e.g., femoral head, tibial plate, patellar plate, and/or meniscus replacement plate), artificial shoulder (e.g., humeral component, stem, and/or glenoid component), artificial arm, artificial finger, or a part thereof. The implants may be fabricated of metal and may include pins, screws, plates, prostheses, nails, rods. In an embodiment, the medical implant is a pin, screw, plate, prostheses, nail, or rod.

The films have a desirable contact angle. For example, the contact angle with deionized water or simulated body fluid is 20 degrees or less. In an embodiment, the film has a contact angle of 0 degrees to 20 degrees (e.g., with deionized water or simulated body fluid), including all integer degree values and ranges therebetween.

The polarized hydroxyapatite film may have a plurality of metal nanoparticles disposed on at least a portion of an exposed surface of the film. The metal nanoparticles can be any metal nanoparticles that can be deposited on the films. Methods of metal nanoparticle formation are known in the art, and include electrochemical reduction of metal ions in an aqueous solution. For example, the metal nanoparticles are silver nanoparticles, magnesium nanoparticles, copper nanoparticles, or platinum nanoparticles. The metal nanoparticles may be a mixture of nanoparticles having one or more different compositions. The nanoparticles can have a wide range of sizes (e.g., a largest dimension such as diameter). For example, the nanoparticles have a size of 1 nm to 200 nm, including all integer nm values and ranges therebetween.

The metal nanoparticles can be disposed on a wide range of surface area (e.g., exposed surface area) of the film. For example, the nanoparticles are disposed on 0.1 to 100%, including all values to the 0.1% and ranges therebetween, of the surface area (e.g., exposed surface area) of the film. For example, the metal nanoparticles are disposed on 100% of the surface area (e.g., exposed surface area) of the film such that the nanoparticles form a continuous layer on the film. For metal nanoparticles formed from electrochemical reduction of metal salts, the amount of surface area covered by the metal nanoparticles can be controlled by selecting the synthesis time and/or metal salt concentration (e.g., silver nitrate concentration). Typically, longer synthesis time and higher metal salt concentration results in higher surface area coverage than shorter synthesis time and/or metal salt concentration. For example, in the case of silver nanoparticles, where 100% of the surface is covered, the nanoparticle size is larger (approximately 100 nm), and a majority of the nanoparticles are fused together, and at the shortest times, there are only a few silver nanoparticles at disposed on the tips of the HA crystals.

In an embodiment, the film has as plurality of silver nanoparticles disposed on at least a portion of surface (e.g., an exposed surface) of the film. Such films exhibit antimicrobial properties. Without intending to be bound by any particular theory, it is considered that the antimicrobial properties result from the release of silver ions from the silver nanoparticles. It is expected that smaller silver nanoparticles release more silver ions than larger silver nanoparticles. Accordingly, in an embodiment, the silver nanoparticles have a size of 100 nm or less. In another embodiment, the silver nanoparticles have a size of 1 nm to 100 nm.

In an aspect, the present disclosure provides methods of making polarized hydroxyapatite films. The methods are based on the electrochemical growth of films of crystals (growing a film on a substrate across which a voltage is applied) or on application of a voltage to an already-formed film. Optionally, additional hydroxyapatite crystals are deposited by hydrothermal methods on the films comprised of electrochemically grown crystals.

The films may be deposited in a single electrochemical growth step. This layer of hydroxyapatite crystals is also referred to herein as a seed layer. The hydroxyapatite crystals deposited during this step are 10 nm to 5000 nm, including all integer nm values and ranges therebetween, in size. The crystals may be rod-shaped crystals.

In an embodiment, the method of making a film disposed on substrate, the film comprising polarized hydroxyapatite, comprises: a) heating a precursor solution comprising a calcium source (e.g., calcium chloride), a phosphate source (e.g., potassium phosphate), and, optionally, pH buffered sodium chloride solution to a temperature of 50° C. to 100° C. (e.g., 90° C.); and b) applying an electrical current at a current density of 1 to 300 mA/cm² (e.g., 12.5 mA/cm²) to the precursor solution after reaching the desired reaction temperature, such that the film comprising polarized hydroxyapatite comprising a plurality of rod-shaped crystals having a crystallographic c-axis normal to the substrate, the film having a residual electrostatic charge of at least 5 mC/cm² is formed. The films deposited by a single step method can have a thickness of 10 nm to 5 microns, including all nm values and ranges therebetween. In an embodiment, the method further comprises contacting the film from b) with a metal salt solution such that metal nanoparticles are formed and disposed on at least a portion of the surface of the film.

The sodium chloride solution is pH buffered. For example, the sodium chloride solution is buffered to a pH of 3.0 to 11.0, including all pH values to 0.1 pH units and ranges therebetween. For example, the sodium chloride solution is buffered to a pH of 7.2.

An electric current is applied to the solution during film formation (e.g., a) and b) above). The current is applied after the desired reaction temperature is reached. The electric current is applied, for example, for 0.25 minutes to 30 minutes, including all integer minute values and ranges therebetween, and during this time a hydroxyapatite film is deposited. For example, the current is applied for 2.5 to 5 minutes. A constant (i.e., fixed) current may be applied (e.g., typically 12.5 mA/cm²) to the precursor solution and the corresponding voltage ranges from 2.0 to 5.0 V. The power supply is allowed to float voltage while holding current fixed. A constant (i.e., fixed) voltage may be applied and the current allowed to float.

In an embodiment, the electric current is applied to the solution after reaching the desired reaction temperature by using the metal substrate as cathode for depositing hydroxyapatite and passing current through the solution to the anode (e.g., a platinum electrode or graphite electrode). The HA thin film is coated on the metal substrate as current is applied during the desired reaction time.

The films deposited by a single step may be heated to alter the polarization of the films. In an embodiment, the film deposited by a single step are heated at 200° C. to 500° C., including all integer ° C. values and ranges therebetween, for 0.5 min to 2 hours, including all hour values to 0.5 and ranges therebetween. In an embodiment, the film deposited by a single step are heated at 200° C. to 500° C., including all integer ° C. values and ranges therebetween, for 0.1 min to 2 hours, including all minute values to 0.1 and ranges therebetween. The films are heated without altering the crystal morphology of the films. For example, films are heated to 500° C. for 24 hours without altering the crystal morphology of the films.

During at least a portion of the heating step, an electric field of 1 kV/cm or greater may be applied to the film. At very high voltages, films can be destroyed by dielectric breakdown. In an embodiment, an electric field of 1 kV/cm to 100 kV/cm, including all integer kV values and ranges therebetween, is applied to the film. The electric field is applied, for example, by applying a DC current to parallel electrodes placed above and below the film. Where the substrate is a conducting metal substrate (e.g., a titanium substrate), the metal substrate is one of the electrodes.

The polarized hydroxyapatite films may be formed by a two-step method. In such methods, first a film is deposited by an electrochemical method as described herein, subsequently, additional hydroxyapatite is deposited on the electrochemically deposited hydroxyapatite by a seeded hydrothermal method (also referred to herein as hydrothermal crystallization). The films deposited by the two-step method can have a thickness of 5 microns to 50 microns, including all micron values and ranges therebetween.

In an embodiment, the two-step method comprises: a) depositing a hydroxyapatite film by an electrochemical method; and b) depositing a plurality of hydroxyapatite crystals by hydrothermal crystallization on the film from a). The hydrothermal crystallization step is carried out at, for example, a temperature of 150° C. to 250° C., including all integer ° C. values and ranges therebetween, for 5 hours to 20 hours, including all integer hour values and ranges therebetween.

During formation of the hydroxyapatite by a hydrothermal method, the hydrothermally-formed hydroxyapatite may be doped. Hydroxyapatite has calcium, phosphate, and hydroxide groups. A, B, or C-type doping is substitution of for calcium, phosphate, or hydroxide, respectively. The hydrothermally-formed hydroxyapatite is C-type doped with F⁻, B-type doped with CO₃²⁻, and A-type doped with divalent and trivalent cations including, for example, magnesium, lead, barium, strontium, cerium, europium, lanthanum, yttrium, and ytterbium, from their respective salts. In an embodiment, hydroxyapatite film formed during deposition by hydrothermal crystallization is doped with cations (e.g., divalent cations and trivalent cations), anions (e.g., monoatomic anions and polyatomic anions), or a combination thereof.

Metal nanoparticles may be deposited on the polarized hydroxyapatite films. The metal nanoparticles are deposited on films formed in a single electrochemical growth step (i.e., one-step method) or films deposited by an electrochemical growth step followed by a hydrothermal growth step (i.e., two-step method).

In an embodiment, the metal nanoparticles are deposited in an electrochemical deposition (i.e., electrochemical reduction of metal ions in an aqueous solution) step. The metal nanoparticles are, for example, formed by contacting the film with a metal salt solution under electrochemical reduction conditions such that metal nanoparticles are formed and disposed on at least a portion of the surface of the film. If a metal substrate is used, the substrate may be used as an electrode in the electrochemical reduction.

A wide range of conditions (e.g., metal salt precursor concentration, reaction time, and reaction temperature) can be used in the electrochemical reduction step. In an embodiment, silver nanoparticles are deposited at a silver nitrate concentration of 0.0001 to 1 M (e.g. 0.00125 M) and sodium chloride concentration of 0.0001 to 1M (e.g., 0.00125M), where the deposition time is 1 second to 1000 seconds (e.g., 90 seconds), the temperature is 20° C. to 90° C., and the voltage is 1 V to 30 V (e.g., 4 V). Complete (100%) coverage of the polarized hydroxyapatite film is obtained at a silver nitrate concentration of 0.025 M.

The crystals can be removed from the substrate by chemical methods or physical methods to provide a plurality of free crystals. For example, the crystals are removed by physical scraping or ultrasound.

The steps of the method described in the various embodiments and examples disclosed herein are sufficient to carry out the methods of making the HA crystals or films thereof of the present disclosure. Thus, in various embodiments, the method consists essentially of a combination of the steps of the methods disclosed herein. In another embodiment, the method consists of such steps.

In an aspect, the present disclosure provides uses of the polarized hydroxyapatite crystals of films thereof. For example, the polarized hydroxyapatite films can be used as a surface on which additional hydroxyapatite can be grown in vitro or in vivo or as a catalyst support.

In an embodiment, a method for growing hydroxyapatite comprises exposing the polarized hydroxyapatite film to body fluid or simulated body fluid such that additional hydroxyapatite is deposited on the film. The methods can be carried out in vitro or in vivo.

In an embodiment, the polarized hydroxyapatite film with nanoparticles disposed on the film is a catalyst. For example, the nanoparticles catalyze chemical reactions.

The steps of the methods described in the various embodiments and examples disclosed herein are sufficient to carry out the methods of using the HA crystals or films thereof of the present disclosure. Thus, in various embodiments, one of these methods consists essentially of a combination of the steps of the methods disclosed herein. In another embodiment, the method consists of such steps.

The polarized hydroxyapatite films can be used in devices. In an embodiment, a device comprises a polarized hydroxyapatite film. The film is disposed on at least a portion of a surface (e.g., external surface) of the device. The film may be disposed on all of the exterior surfaces of the devices. The film may have a plurality of metal nanoparticles disposed on at least a portion of a surface of the film. For example, the metal nanoparticles are silver nanoparticles that can be an antimicrobial coating. Examples of suitable devices include medical implants, electrets, and filters. Devices comprising a polarized hydroxyapatite film with metal nanoparticles disposed on the film, e.g., silver nanoparticles, can be an antimicrobial coating.

The device may be a medical device such as, for example, a medical implant. In an embodiment, the device is a medical implant comprising a polarized hydroxyapatite film. A medical implant comprising the polarized hydroxyapatite film can provide rapid bone (e.g., hydroxyapatite) growth and osseointegration. The medical implants can be, for example, orthopedic implants, which may be used to support a damaged bone or to replace a missing joint or bone. Orthopedic implants can be used with or to replace or at least partially replace various parts of the skeletal system, including, for example, a hip, spine, neck, elbow, femur, knee, shoulder, arm, or finger. The implants, which may be fabricated of metal, may include pins, screws, plates, prostheses, nails, rods, or other devices. For example, the medical device is an orthopedic implant such as an artificial hip (e.g., socket and/or ball), artificial spine, artificial neck, artificial elbow, artificial femur, artificial knee (e.g., femoral head, tibial plate, patellar plate, and/or meniscus replacement plate), artificial shoulder (e.g., humeral component, stem, and/or glenoid component), artificial arm, artificial finger, or a part thereof. The implants may be fabricated of metal and may include pins, screws, plates, prostheses, nails, rods. In an embodiment, the medical implant is a pin, screw, plate, prostheses, nail, or rod.

In an embodiment, the polarized hydroxyapatite films can be used as a surface on which hydroxyapatite can be grown in vitro or in vivo.

Electrets comprising the polarized hydroxyapatite film can be used in micropower generation and storage (e.g., in harvesting vibration energy). A filter comprising the polarized hydroxyapatite film can be, for example, ion-exchange type filters (e.g., for liquid solution filtration), and electrostatic filters (e.g., for air purification).

The following examples are presented to illustrate the present disclosure. They are not intended to limiting in any manner.

### Example 1

This example describes preparation and characterization of polarized hydroxyapatite films.

In this example, it was demonstrated that strong electrical polarization is retained in HA coatings on titanium after electrochemical synthesis from aqueous solution at relatively low temperature. The electrical polarization is a result of the local field gradient induced near the titanium electrode during synthesis. The stored charge is larger by more than an order of magnitude than any previously reported for HA, and is the highest reported for any electret material. The polarized HA coatings on titanium display improved bioactivity, indicating promising potential in orthopedic implants. The very high stored charge may enable new applications of the HA coatings in electret generators, filters or energy storage.

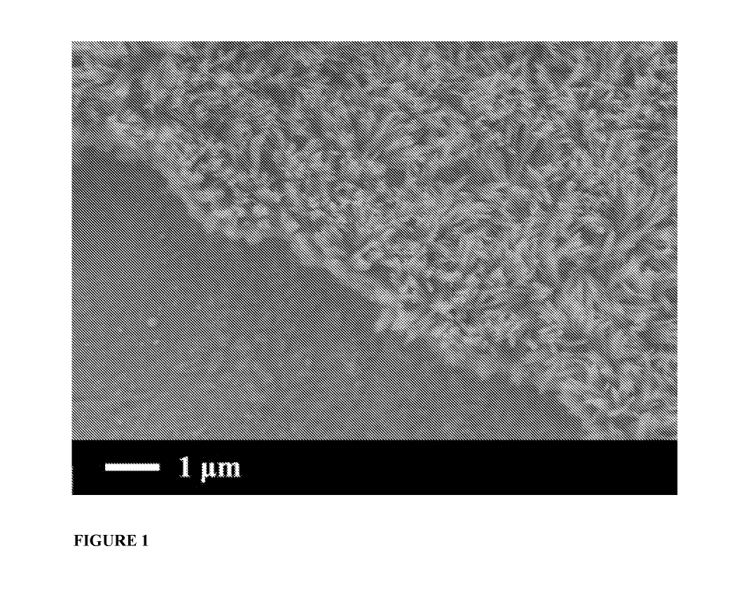

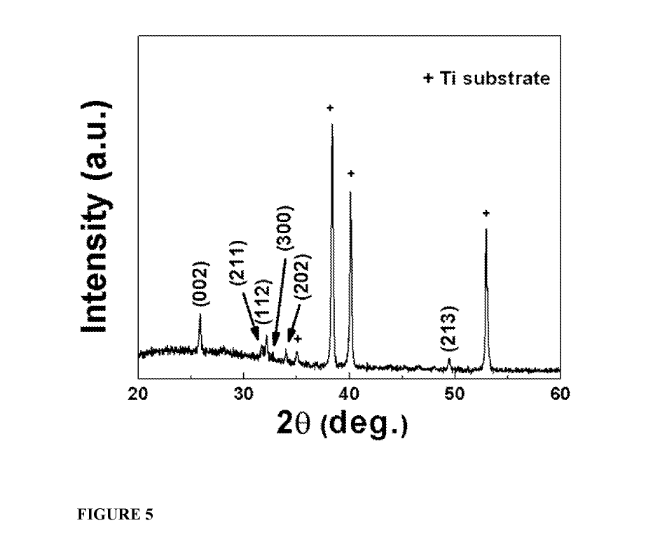

Electrochemical growth of HA is a well-established method to produce uniform, crystalline coatings at relatively low temperature from aqueous electrolyte solutions. In this technique, the metal surface to be coated acts as the cathode and is separated from an anode (typically platinum or graphite) in a simulated body fluid electrolyte solution. When an electric potential is applied, the polarization of the electrodes causes the local ion concentration near the electrode surface to deviate from that in the bulk. Cations, such as Ca²⁺, are attracted to the cathode surface, while anions are repelled from the cathode and attracted to the anode. The voltage across the electrodes (typically 3-4 volts) is sufficient to induce electrolysis of water and therefore cause a local increase in pH near the cathode surface. As a result of changes in pH and electrolyte concentration near the cathode surface, HA becomes locally supersaturated and nucleates selectively on the cathode. FIG. 1a shows HA nanocrystals formed on the titanium surface after a reaction time of 5 minutes at 95° C. The crystals are needle-like with the long axis associated with the crystallographic c-axis. In the image, a section of the coating has been scraped away to show the underlying titanium surface, and preferential orientation of the rod-shaped crystals normal to the surface is apparent. The coating is approximately 500 nm thick, and X-ray diffraction (FIG. 5) confirms that it consists of HA with a preferential orientation of the c-axis normal to the substrate.

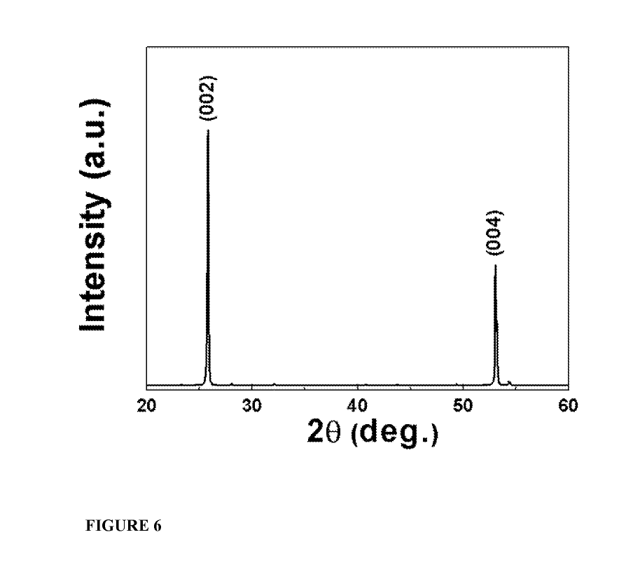

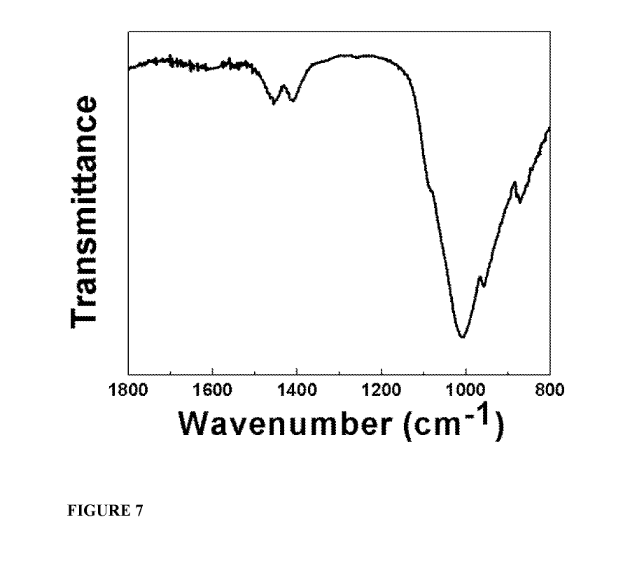

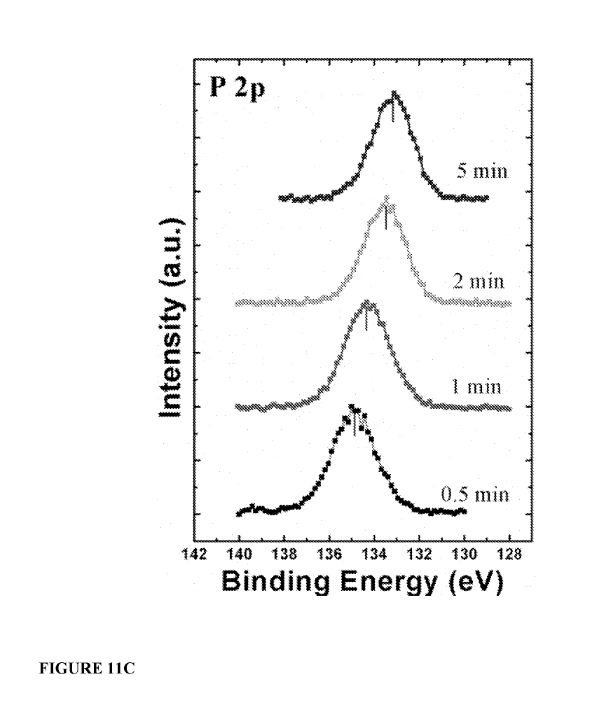

The HA nanocrystals shown in FIG. 14A were used as seeds to promote growth of additional HA on the surface during hydrothermal crystallization. Urea added during the hydrothermal synthesis induces the formation of a dense coating, and results in B-type carbonate ion substitution in which a fraction of the phosphate groups in HA are replaced by carbonate groups. FIG. 14B shows a side view image of the resulting carbonated HA coating that is approximately 10 microns thick. X-ray diffraction of the dense sample (FIG. 6) confirms it is comprised exclusively of HA with near perfect alignment of the c-axis normal to the substrate. FTIR spectroscopy (FIG. 7) confirms B-type carbonate ion substitution, and elemental analysis shows a carbonate content of ˜6%. The composition is similar to that of natural apatite in bone that has B-type carbonate substitution with 4-6% carbonate content.

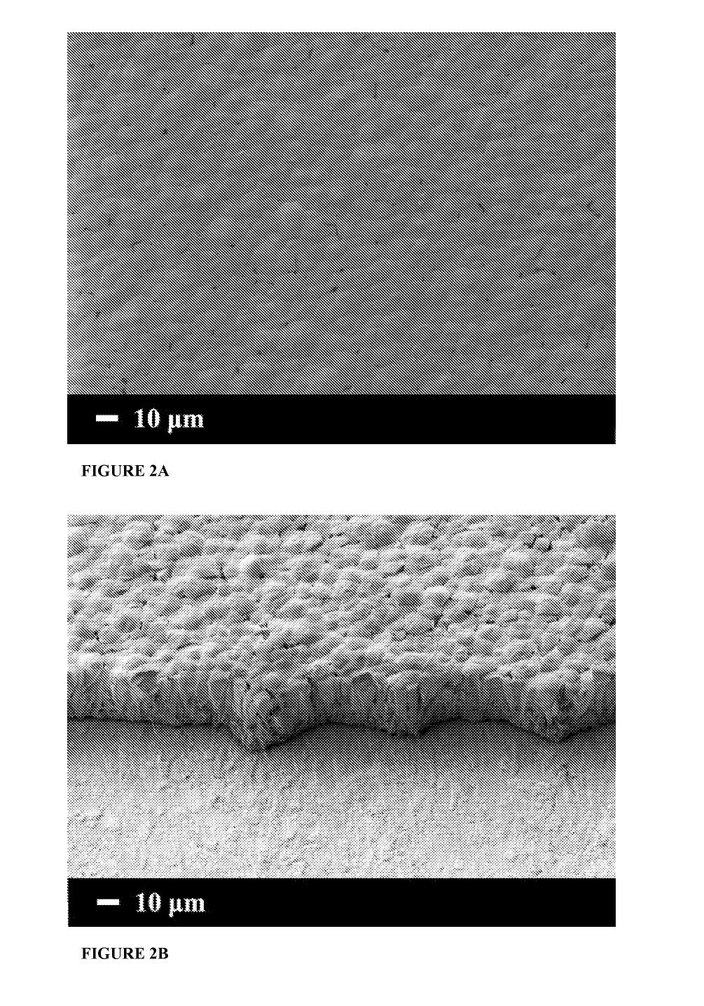

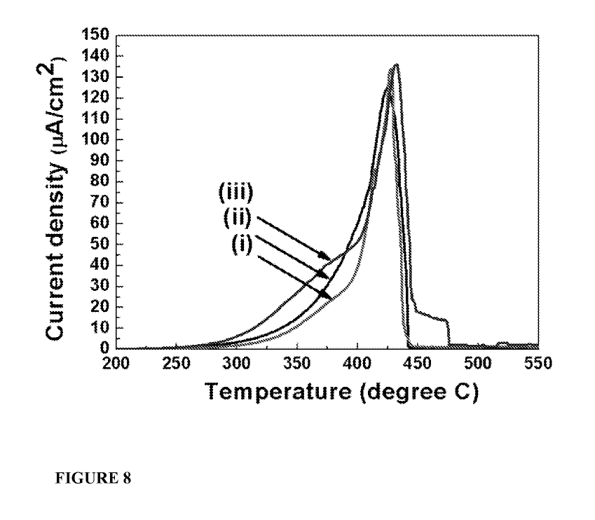

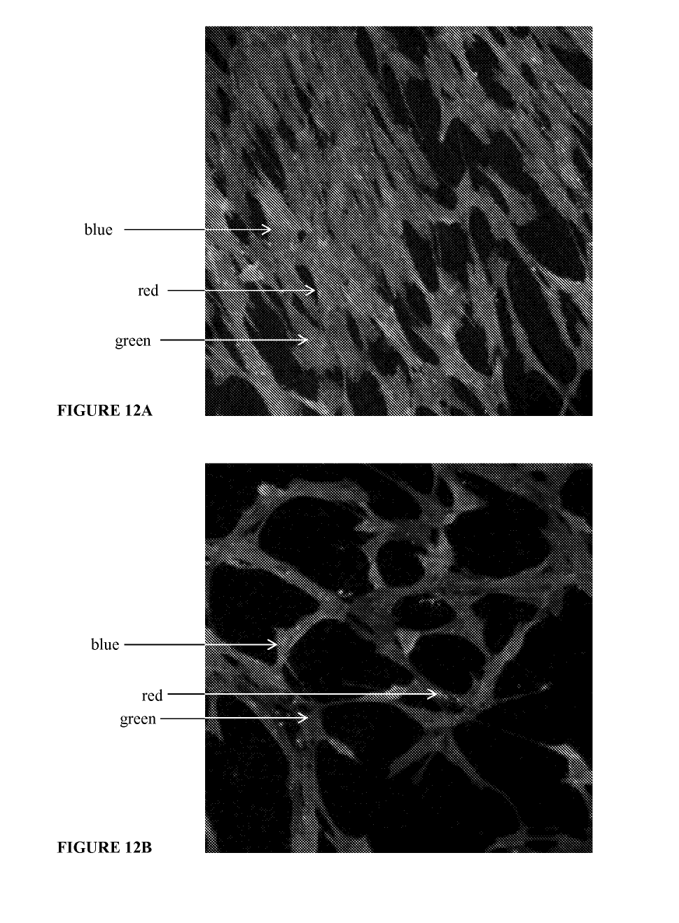

The stored electrical charge in the as-synthesized carbonated HA coating was measured using the thermally stimulated depolarization current (TSDC) technique. In this method, electrodes are attached to both sides of the coating and electrical current is measured as the sample is heated to relax polarization via ion transport. FIG. 15 shows the measured current density in microamps per square centimeter versus temperature for a sample that was heated at a rate of 5° C. per minute. A surprisingly high peak current density of 126 μA/cm² was measured at 425° C. The current density falls sharply to zero as the sample is heated above 425° C., indicating that the sample was completely depolarized. The total stored charge (Q) is obtained by integrating the current, using the formula:

$$Q = \frac{1}{β} \int J ( T ) dT$$

where β is the heating rate and J(T) is the current density at temperature T. The data in FIG. 2 give a total stored charge of 70 mC/cm². The TSDC was repeated several times on different samples, each giving similar results (FIG. 8). The highest previously published stored charge for HA is ˜4 mC/cm² for a carbonated sample after being polarized under an electric field strength of 2 kV/cm for 30 minutes at 350° C. The very large stored charge shown in the data of FIG. 2 is unexpected since the sample was never heated under an applied field to induce polarization by ion transport.

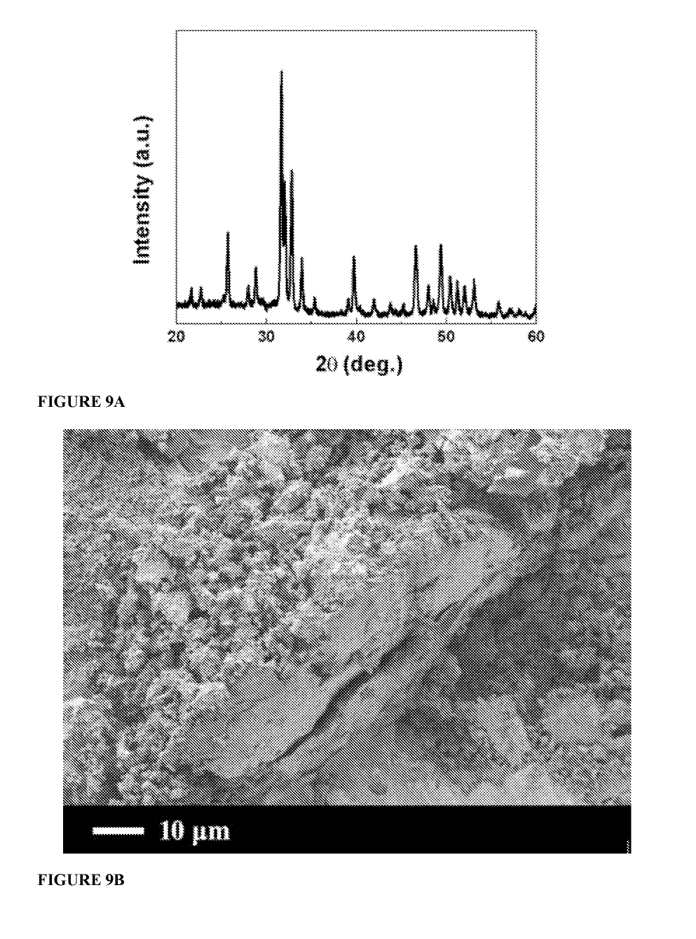

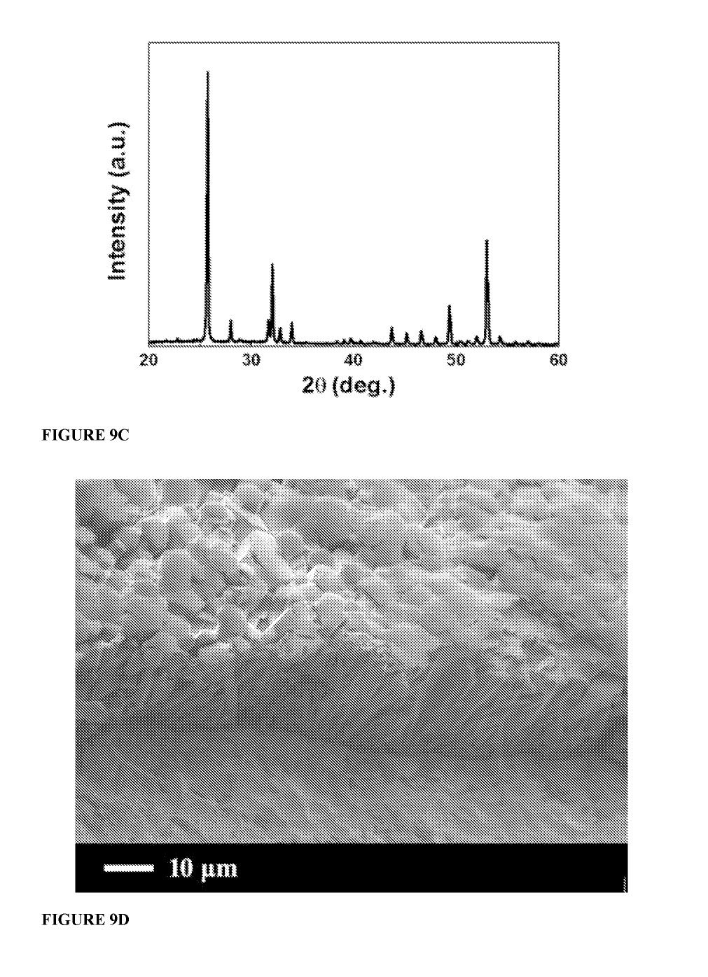

We hypothesize that the stored charge arises during the electrochemical synthesis of the HA seed crystals, as it is the only synthesis step in which an external field is applied. Unfortunately, direct measurement of the stored charge in the seed layer by TSDC is not possible. Electrodes placed directly on the seed layer cause short circuiting due the thin and porous morphology of the coating (FIG. 14A). Good electrode contact for TSDC measurement requires a dense HA coating of relatively uniform thickness. For HA deposited onto titanium by plasma spray, TSDC measurement showed zero stored charge as expected (FIGS. 9A-D and FIG. 10). In a second control experiment, TSDC measurement was carried out on a dense carbonated sample similar in structure to that shown in FIG. 14b, except that seed crystals were deposited on titanium through evaporative deposition from colloidal suspension rather than electrochemically. The stored charge was measured to be only 0.5 mC/cm² for the control sample (FIGS. 9A-D and FIG. 10), with two small current peaks consistent with proton migration and space charge polarization induced by dehydroxylation. The dehydroxylation process is likely asymmetric due to the anisotropic structure of the sample. While dehydroxylation is responsible for some of the measured charge, it does not account for the very high stored charge measured in FIG. 15.

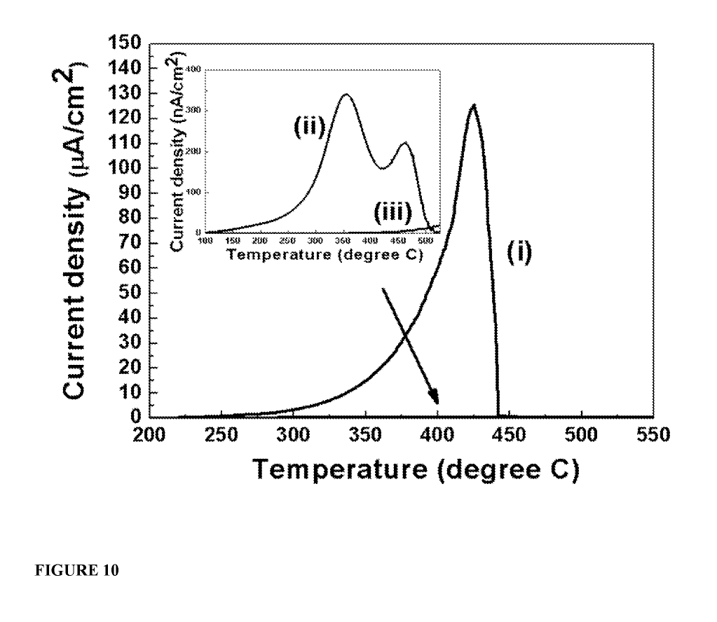

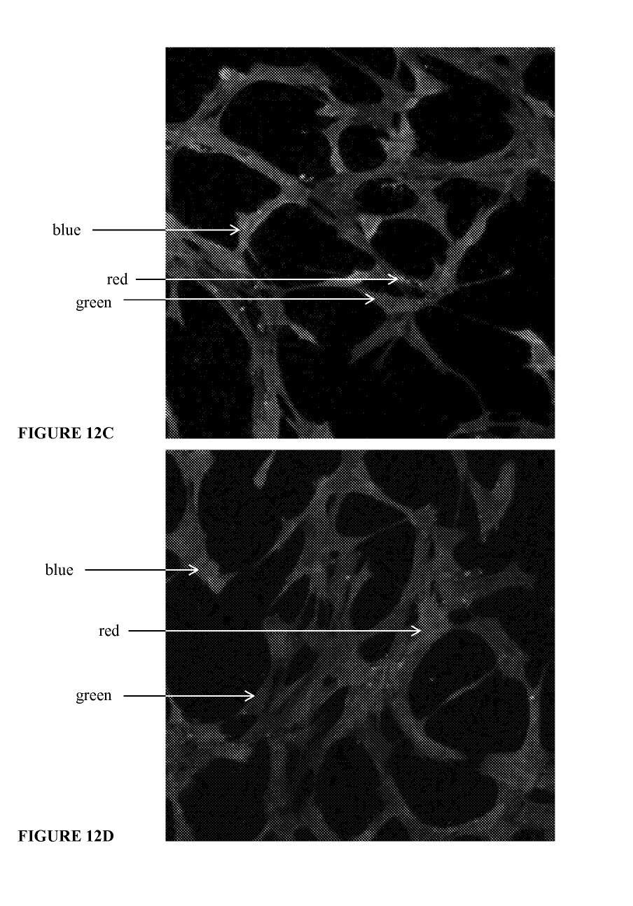

FIGS. 16A-C illustrate the proposed mechanism of polarization during electrochemical synthesis of HA. The nucleation and growth of the HA nanocrystals at the titanium surface involves the reaction of calcium, phosphate, and hydroxyl ions within the electrical double layer of the electrolyte solution at the titanium surface. The negatively charged titanium cathode attracts calcium ions, so that the HA that initially nucleates on the surface is calcium-rich. As the dielectric HA layer grows thicker, it partially shields the electric field so that the local field experienced by ions in the adjacent electrolyte solution decreases. These ions are immobilized in the solid HA phase as it grows. As a result, there is a gradient in concentration of positive calcium ions relative to negative phosphate and hydroxyl ions in the HA versus distance from the titanium surface. The electrical charge distribution in the solid coating is due to the ion distribution in nonstoichiometric HA. The TSDC data (FIG. 15) show that appreciable ion movement does not occur at temperatures below ˜225° C. As a result, a quasi-permanent electrical dipole (illustrated by the arrow in FIG. 16C) is maintained in the HA layer at lower temperatures. Supporting evidence of the postulated charge distribution was obtained from X-ray photoelectron spectroscopy (XPS) on the surface of HA electrochemically grown for 0.5, 1, 2, and 5 minutes (FIGS. 11A-C). The XPS results show that the ratio of calcium to phosphorous (Ca/P) at the surface decreases from 1.67 to 1.60 as the synthesis time increases from 0.5 to 5 minutes. XPS measurement also revealed an increase in the surface concentration of hydroxyl groups with increasing synthesis time. The XPS results are consistent with the mechanism illustrated in FIGS. 16A-C, and reveal that the HA surface becomes progressively more calcium deficient as it grows thicker resulting in excess negative charge on the HA surface and excess positive charge at the HA/titanium interface.

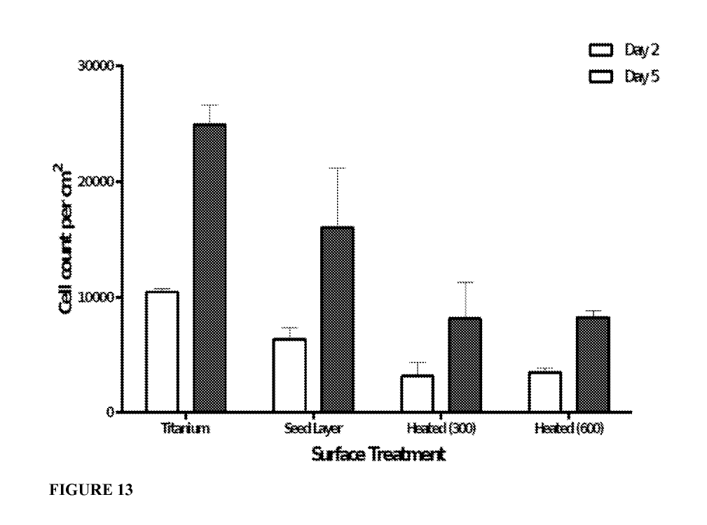

Supporting evidence of strong stored charge was obtained through measurement of in vitro growth of additional HA onto the coating from simulated body fluid. It is known that negative charge on the surface of HA promotes growth of HA from simulated body fluid, while positive surface charge retards growth. FIGS. 17A-F show the surface of the coating after being placed in simulated body fluid for 24 hours at 37° C. A new porous HA layer grew onto the surface of both the seed layer and carbonated coating. FIGS. 17A and 17B show top and side views, respectively, of the new HA deposited onto the seed layer (shown in FIG. 14A). The rod shaped seed crystals are still visible at the bottom of the coating in FIG. 17B. FIGS. 17C and 17D show the top and side views, respectively, of the new HA deposited onto the carbonated coating. In FIG. 17D, the interface between the carbonated HA coating and the new porous HA layer is clearly visible. The morphology of the HA deposited from simulated body fluid is similar for both samples. However, the new HA layer is much thicker on the seed crystals than on the carbonated HA, which is consistent with the expected higher surface charge on electrochemically grown HA than on the hydrothermally grown HA. The dense coating was fractured and pieces carefully removed from the titanium. FIG. 17E shows the bottom surface of HA that was originally at the titanium/HA interface. No additional HA grew onto this surface from simulated body fluid, as shown in FIG. 17F. The result is consistent with the HA surface having strong positive charge at the titanium/HA interface, as positive surface charge is known to suppress HA nucleation and growth.

The depolarization current and simulated body fluid results confirm that electrochemically grown HA coatings retain significant stored charge after synthesis due to quasi-permanent electrical polarization. The polarization direction is such that dipoles are aligned with the negative charge on the exterior surface. The finding is significant due to the fact that negative surface charge has been shown to enhance bone growth in vivo.

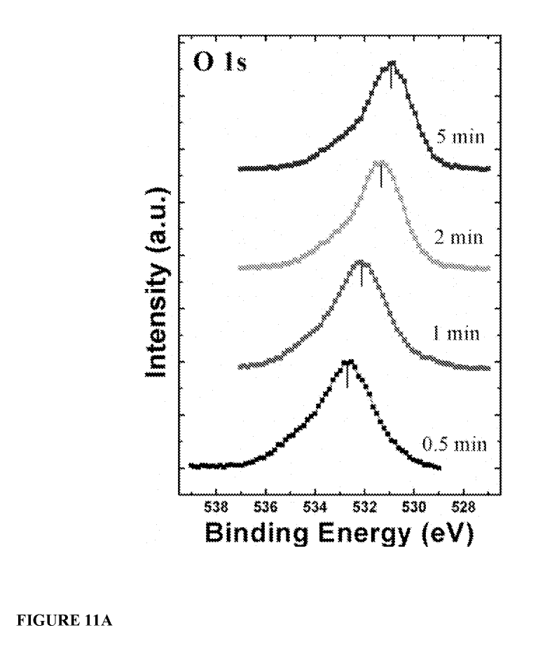

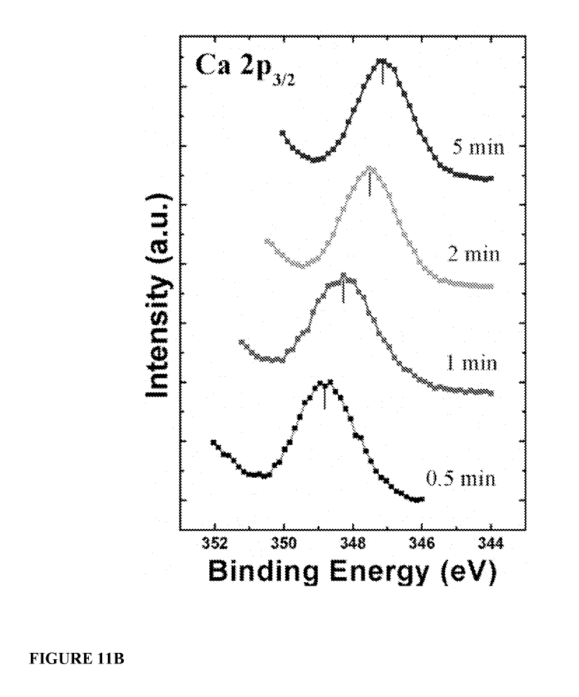

Preliminary in vitro study has revealed that osteoblast cells adhere and proliferate on the strongly polarized coatings (FIGS. 12A-D and FIG. 13). The polarized coatings may offer a novel route to stimulate bone growth around orthopedic and dental implants, providing new treatment options to patients with poor bone quality or bone function due to underlying medical conditions such as osteoporosis, diabetes, and impaired immune system. The large magnitude of the stored charge may also enable new uses of hydroxyapatite in energy storage, ion exchange membranes, or electret micropower generators.

Methods Summary. The electrochemical synthesis of the seed crystals and hydrothermal synthesis of carbonated HA was carried out following the procedure recently reported. The HA samples were characterized by X-ray diffraction (XRD, PW3020, Philips) with Cu Kα radiation (λ=1.5418 Å), scanning electron microscopy (SEM, DSM982, Zeiss-Leo), elemental analysis (CE-440, Exeter Analytical, Inc.), and Fourier transform infrared (FTIR) spectroscopy (FTIR-84005, Shimadzu). For TSDC measurements, the upper surface of the carbonated HA coating was then sputter coated with ˜10 nm platinum. Two Pt foil electrodes were attached to the coating and Ti substrate and platinum leads were used for current measurement. Samples were first heated to 300° C. in air for 1 hour to remove any surface contamination prior to TSDC measurement. The samples were then heated at a rate of 5° C./min in a tube furnace (Lindberg Blue M, Thermo Scientific) as current was measured using a picoammeter (Model 6487, Keithley Instruments).

The surface composition was obtained with an XPS spectrometer (SSX-100, Surface Science Laboratories), equipped with a monochromatic A1 anode x-ray gun (Kα=1486.6 eV). The base pressure of the system was 1×10-11 torr. The spot size of the X-ray was chosen to be 1 mm in diameter and the resolution selected for energy analyzer was 0.5 eV. The elemental composition of the samples was determined from spectra of the O-1s, Ca-2p and P-2p core levels. Since hydrogen could not be detected by XPS, the hydroxide concentration was determined from the O-1s spectra. Selected HA samples immersed into a 5 ml solution of 1.5× simulated body fluid (SBF) with pH=7.25.¹⁸ After 24 hours in SBF at 37° C., the samples were taken out of the solution and placed in a desiccator. New HA grown from SBF was then characterized by SEM and XRD.

### Example 2

This example describes preparation and characterization of polarized hydroxyapatite films.

Production of coatings of hydroxyapatite (HA) that retain significant electrostatic charge was demonstrated. The coatings are prepared on metal substrates through a single electrochemical synthesis step or through a two-step electrochemical/hydrothermal deposition method. The HA crystals are polarized during the electrochemical synthesis step, or optionally by placing the coating in an electric field at elevated temperature to facilitate proton transport in the crystals. The polarization of the crystals significantly enhances the deposition of additional HA from simulated body fluid in vitro. It is expected that the polarized coatings will enhance bone growth in vivo.

The method involves the following steps:

(1) Electrochemical crystallization of HA following a similar method reported in our previous patents and journal articles. This synthesis step is carried out using a solution of calcium chloride, potassium phosphate, and optionally sodium chloride buffered to pH 7.2. The reaction is carried out typically at 95° C. under constant current density for ˜2-20 minutes. The voltage applied during deposition is typically 3-4 volts. The resulting coatings consist of rod shaped crystals preferentially oriented with the crystallographic c-axis normal to the substrate, as shown in FIG. 1. The thickness of the coating is typically near 1 micron. We discovered that the elimination of sodium chloride from the synthesis solution did not significantly affect crystallization, but allowed a slightly higher voltage to be applied in order to enhance polarization of the deposited crystals. The thickness of the coating can be adjusted by varying the reaction time and concentration of reagents.
(2) A hydrothermal crystallization may optionally be carried out to create a dense coating. The hydrothermal crystallization is similar to that reported in our previous work. During hydrothermal crystallization, the electrochemically deposited HA acts as seed for additional crystal growth. The crystals may be doped during hydrothermal crystallization with a variety of cations or anions, including fluoride, carbonate, magnesium, yttrium, ytterbium, europium, strontium, and cerium. The hydrothermal crystallization allows for a thicker and more dense coating, while maintaining preferential orientation of the crystallographic c-axis normal to the substrate.
(3) Post synthesis thermal processing. The coating is heated to enhance polarization. The thermal processing step may be carried out with or without an applied electric field. It was discovered that heating to moderate temperatures (˜300° C.) for ˜1 hour would enhance measured polarization by enabling better electrode contact, while high temperature heating (˜>600° C.) would reduce polarization by ion transport. The coating can also be heated while applying an electric field of >1 kV/cm in order to recover some of the polarization by proton transport.

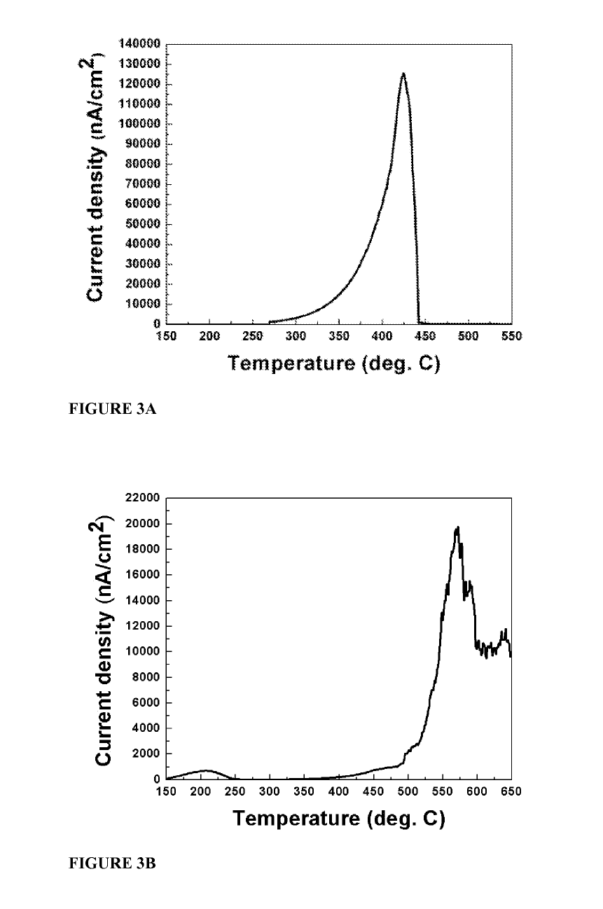

Characterization of polarization. The stored electrostatic charge is measured through a thermally stimulated depolarization current (TSDC) measurement. In this technique, the titanium substrate serves as one electrode, and a platinum counter electrode is applied via sputter coating the HA surface. An ammeter is applied to the electrodes and current flow is recorded as the sample is heated. By integrating the total current released as polarization is dissipated, the stored charge is obtained. The TSDC method is a well-established technique for characterizing stored charge, including stored charge in HA. FIGS. 3A-B show the TSDC results for a carbonated HA sample (shown in FIGS. 2A-B) synthesized by electrochemical/hydrothermal method (FIG. 3A) and an HA sample synthesized solely by the electrochemical method for 20 minutes (FIG. 3B). The carbonated HA sample has a peak current density of 126 μA/cm² at 425° C. (FIG. 3A). The stored charge is 70 mC/cm² which is an order of magnitude higher than the largest value previously measured for HA. The electrochemically deposited HA has a peak current density of 20 μA/cm² at 573° C. (FIG. 3B). The stored charge is 25 mC/cm². The sample used in FIG. 3B is not dense, so platinum could not be sputter coated on the surface without short circuiting the electrodes. Instead, a platinum foil was used as the electrode to collect the data in FIG. 3B. As a result, there is poor contact and significant noise in the data. However, the data show significant stored charge in the sample produced by electrochemical deposition alone.

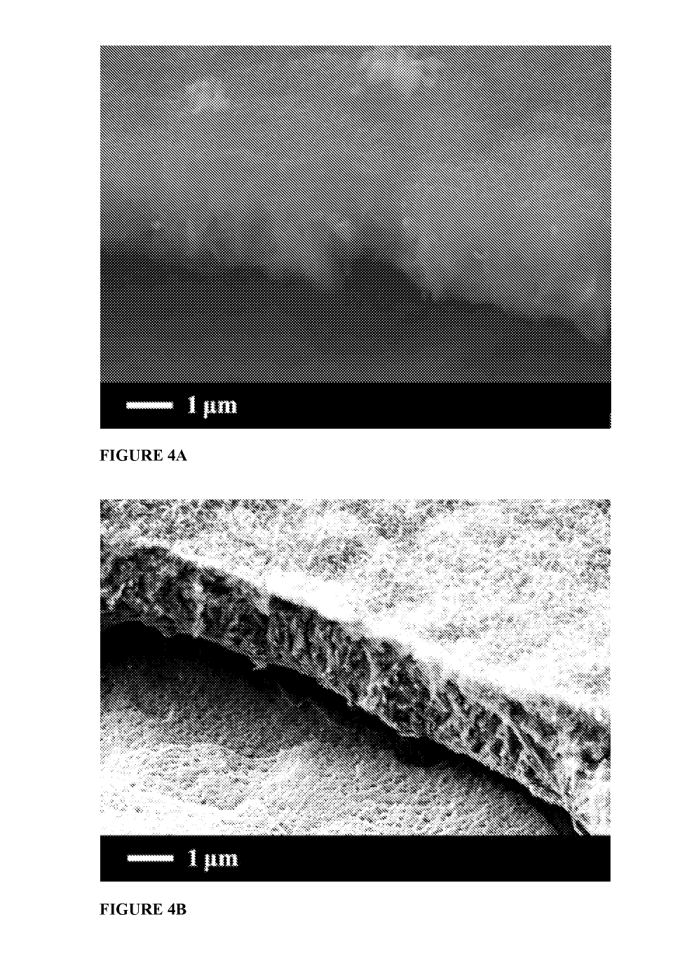

Additional HA growth onto polarized HA from simulated body fluid. The polarized HA coating was placed in 1.5× simulated body fluid for 24 hours at 37±1° C. to examine the effect of polarization on the deposition of additional HA from solution. Two samples were used, both of which had HA deposited by electrochemical deposition for 5 minutes. In one sample, the coating was used as synthesized. In the other, the coating was heated to 600° C. to relax the polarization. FIG. 4 shows the images of the samples from electron microscopy. In FIG. 4A it can be seen that a new layer of porous HA, approximately 1 micron thick, is deposited on top of the polarized rod shaped crystals. In FIG. 4B, a much thinner HA layer, approximately 250 nm thick, is deposited on the sample that has been heated to 600° C. to depolarize the sample. The results confirm that the polarized coating effectively enhances the growth of additional HA from simulated body fluid. The in vitro results are a promising indication that the polarized coatings may enhance bone growth in vivo.

### Example 3

This example describes preparation and characterization of polarized hydroxyapatite films.

In this example, a simple method for synthesizing a bioactive HA coating with antimicrobial silver nanoparticles is presented (AgHA). The initial HA layer was deposited onto a titanium substrate using an electrochemical method. By shortening the electrochemical deposition time, a uniform and thin film of HA crystals was formed. Then, an electrolyte solution containing silver ions was used to electrochemically reduce silver nanoparticles directly onto the HA crystals. The HA and AgHA films were characterized and tested for antimicrobial capabilities.

Experimental. Materials. Titanium (Ti) substrates (12.5 mm×12.5 mm and 0.89 mm thick), platinum foil (25 mm×25 mm and 0.127 mm thick), AgNO₃, MgCl₂.6H₂O (99.0-102% purity) and NaHCO₃ (99.7-100.3% purity) were purchased from Alfa Aesar. K₂HPO₄ (99.99% purity), K₂HPO₄.3H₂O (99% purity), CaCl₂.2H₂O (99+% purity), NaCl (>99.0% purity), tris(hydroxymethyl)-aminomethane (tris) (99.8+% purity), and tryptic soy broth were all obtained from Sigma-Aldrich. CaCl₂ (99.5% purity) and Na₂SO₄ (100.1% purity) was purchased from J. T. Baker. KCl (99.7% purity), 36.5-38.0% hydrochloric acid, and 28.0-30.0% pure ammonium hydroxide were purchased from Mallinckrodt Chemicals. Detergent powder was purchased from Alconox. Deionized (DI) water was used for all solutions.

Electrochemical deposition of HA seeds. Titanium substrates were gently polished with SiC paper (800 grit) to provide surface roughness. The substrates were then thoroughly washed with Alconox detergent powder, rinsed with tap water, attached to a silver wire by tightly wrapping the wire through a premade hole in the substrate, sonicated in an ethanol/acetone (volume ratio=50/50) solvent for 30 minutes, and then rinsed with deionized water. An electrolyte solution was prepared containing 138 mM NaCl, 50 mM Tris, 1.25 mM CaCl₂, and 0.828 mM K₂HPO₄ in 125 mL deionized water per substrate. The solution was buffered to pH 7.2 using hydrochloric acid. The electrolyte solution was then heated to 95° C. The substrate and a platinum plate were held parallel to each other with a fixed distance of separation of 10 mm. The substrate and platinum plate were connected to the negative and positive electrodes of a direct current power supply (Instek GPS-3030D), respectively, and immersed in the electrolyte solution. The electrochemical reaction was carried out for 5 minutes at 12.5 mA/cm² (area relative to the platinum plate), then rinsed off with deionized water and dried in air.

Electrochemical reduction of Ag nanoparticles. For each sample coated with Ag nanoparticles, an 80 mL solution containing 1.25 mM Ag(NO₃) and 1.25 mM NaCl was prepared. The electrolyte solution was then heated to 95° C. The HA coated substrate and a platinum plate were held parallel to each other with a fixed distance of separation of 10 mm. The substrate and platinum plate were connected to the negative and positive electrodes of a direct current power supply (Instek GPS-3030D), respectively, and immersed in the electrolyte solution. The electrochemical reaction was carried out for 90 seconds at 12.5 mA/cm² (area relative to the platinum plate), then rinsed off with deionized water and dried in air.

Bacteria growth testing. A solution of Staphylococcus aureus (S. aureus) (ATCC 25923) bacteria in tryptic soy broth was grown overnight with shaking at 37° C. The bacteria concentration was then diluted with tryptic soy broth until the solution's absorbance value at 490 nm was 0.1. Bacteria growth curves were produced by placing n=3 HA and AgHA samples into wells of a 24 well plate. Each well was then filled with 2 mL of bacteria suspension, as well as three control wells with just bacteria suspension. The samples were then incubated at 37° C. Bacteria growth was measured at various times by placing 200 μL of bacteria solution into a 96 well microtitre plate using light scattering at 490 nm. (BioTek FLx800 Fluorescence Microplate Reader). The 200 μL solution was then placed back into the solution of their respective samples after each measurement.

Simulated body fluid growth. N=3 HA, AgHA, and titanium substrates were placed facing up in plastic tubes containing 15 mL of simulated body fluid that was prepared as described by Kokubo. The chemicals used, their amounts, and order in which they were added is listed in Table 1. The final pH was adjusted to 7.4 with Tris. The solution was kept at 37° C. and the samples were left for 24 hours.

TABLE 1
Recipe for simulated body fluid.

| | | | |
|---|---|---|---|
| #0 | DI H₂O | 750 | mL |
| #1 | NaCl | 11.994 | g |
| #2 | NaHCO₃ | 0.525 | g |
| #3 | KCl | 0.336 | g |
| #4 | K₂HPO₄•3H₂O | 0.342 | g |
| #5 | MgCl₂•6H₂O | 0.458 | g |
| #6 | 88 mL 36.5-38% HCl | 60 | mL |
| | diluted to 1,000 mL | | |
| | with DI H₂O | | |
| #7 | CaCl₂ | 0.417 | g |
| #8 | Na₂SO₄ | 0.107 | g |
| #9 | (CH₂OH)₃CNH₂ (Tris) | 9.086 | g |

| | | |
|---|---|---|
| #10 | DI H₂O | Fill beaker to 1,000 mL |

Sample characterization. Crystal morphology was examined using a scanning electron microscope (SEM, Zeiss-Auriga) The crystal structure was determined by X-ray diffraction (XRD) (Philips PW3020) with Cu K1 radiation (λ=1.540560 [symbol] ) from 20-60° with a step rate of 0.02 degrees/second. Phase identification was made by comparison with the JCPDS files. The composition of HA membranes was determined by EDX (EDAX). Three spots on two different samples were probed at an accelerating voltage of 15 kV and the values were averaged and standard deviations were calculated. For zeta potential measurements, approximately 1 mg of HA sample was scrapped off and placed into 1 mL of DI water. A 90 Plus Particle Size Analyzer by Brookhaven Instruments Corporation was used. 10 runs, with 5 cycles/run were used to measure the zeta potential. The Smoluchowski model was used for calculating zeta potential values.

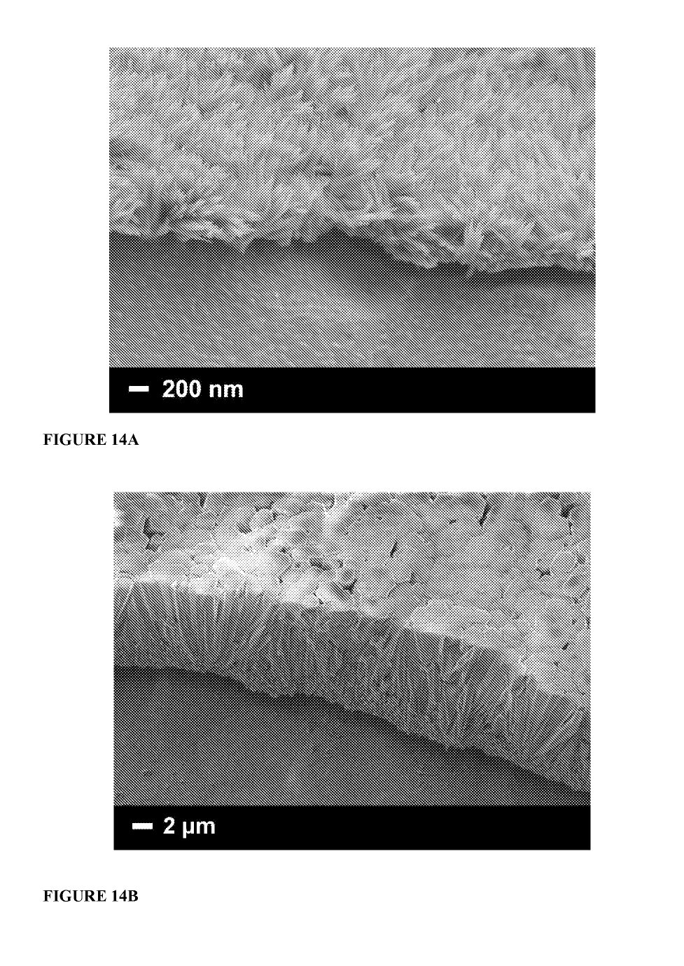

Results and discussion. FIGS. 18A-E show SEM images of the HA and AgHA samples. Individual HA crystals are ˜800 nm long and ˜30 nm wide. For AgHA, the Ag nanoparticles are evenly distributed over the entire HA coating and along each individual HA crystal. The size of the Ag nanoparticles varies from 5 to 50 nm. The variation in nanoparticle size can potentially be advantageous to control the release of Ag⁺ since the rate of ion release is proportional to particle surface area. A range of particle sizes can insure that there will be some Ag⁺ released quickly from the smaller particles, while the larger particles will release Ag⁺ more slowly. Also, according to the Kelvin equation, particles in the nano-size range have a higher solubility limit than the bulk, allowing for a higher amount of Ag delivered if desired.

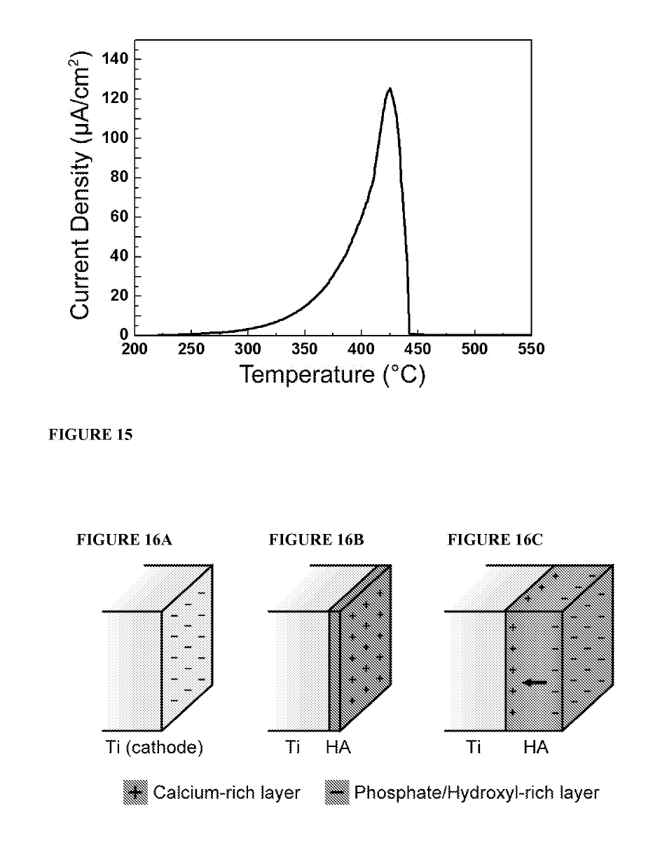

EDX results in Table 2 and FIG. 19 confirm the presence of Ag in the AgHA samples and give an approximate amount of 1.50 At %. While there is a measurable amount of NaCl on the HA sample, most of it is removed after the Ag electrochemical deposition.

TABLE 2
EDX of HA and AgHA.

| Element (At %) | HAP | AgHAP |
|---|---|---|
| Ca | 17.55 ± 0.39 | 19.37 ± 1.36 |
| P | 12.70 ± 0.33 | 13.49 ± 0.76 |
| O | 57.26 ± 1.90 | 65.65 ± 2.11 |
| Ag | — | 1.50 ± 0.27 |
| Na | 7.29 ± 1.10 | — |
| Cl | 5.20 ± 0.74 | — |

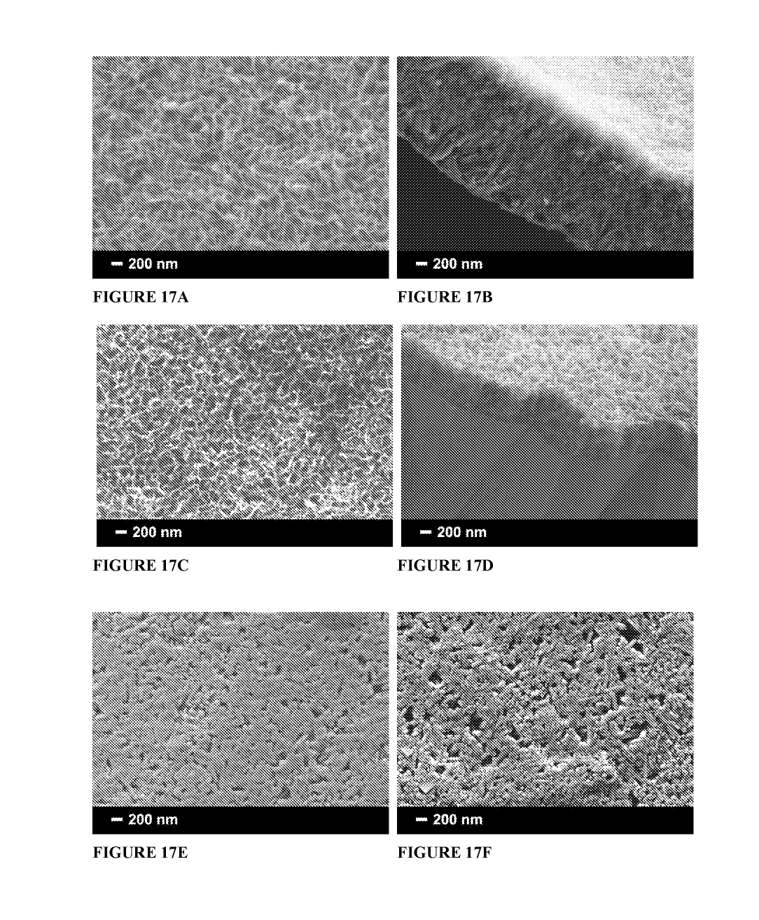

XRD in FIG. 20 verifies that the silver electrodeposition does not change the crystal structure of the HA. The peaks for HA and AgHA match standard HA and titanium according to JCPDS 09-0432 and 01-1197, respectively. HA has an enhanced (0 0 2) peak, showing some preferential orientation along the c-axis. The silver could not be detected using XRD since the highest peak for silver is at 38.117° according to JCPDS 4-0783, which overlaps with the (0 0 2) peak for titanium.

The formation of silver nanoparticles onto HA crystals can occur via a variety of mechanisms. With the applied potential being much larger than the reduction potential of the metal, nonequilibrium conditions are relevant here. The silver ions are reduced in solution according to the equation:

Ag⁺+e⁻→Ag (1)

The positively charged Ag⁺ is attracted to the titanium substrate since titanium is attached to the negative electrode during electrochemical deposition. As the Ag⁺ forms Ag nanoparticles, the nanoparticles may become lodged into the HA crystals due to Ag⁺'s electrostatic attraction toward the titanium. Even the HA crystals themselves inherently have a negative surface charge. The HA crystals have a zeta potential of −15.60±0.51. This is a common result since HA is thought to have an excess concentration of PO₄³⁻ groups at its surface.

Another mechanism could occur via deprotonation of the HA hydroxyl group by a base B:

HAP-OH+B→HAP-O⁻+BH (2)

This step would be followed by an electrophilic attack of Ag⁺:

HAP-O⁻+Ag⁺→HAP−Ag (3)

Also, a very similar mechanism could occur directly onto the titanium substrate:

Ti—OH+B→Ti—O⁻+BH (4)

Ti—O⁻+Ag⁺→Ti—Ag. (5)

Finally, some Ag⁺ can directly substitute for Ca²⁺ in the HA crystal structure to form Ca₅₋ₓAgₓHₓ(PO₄)₃OH, where the extra hydrogen is necessary to maintain charge neutrality.

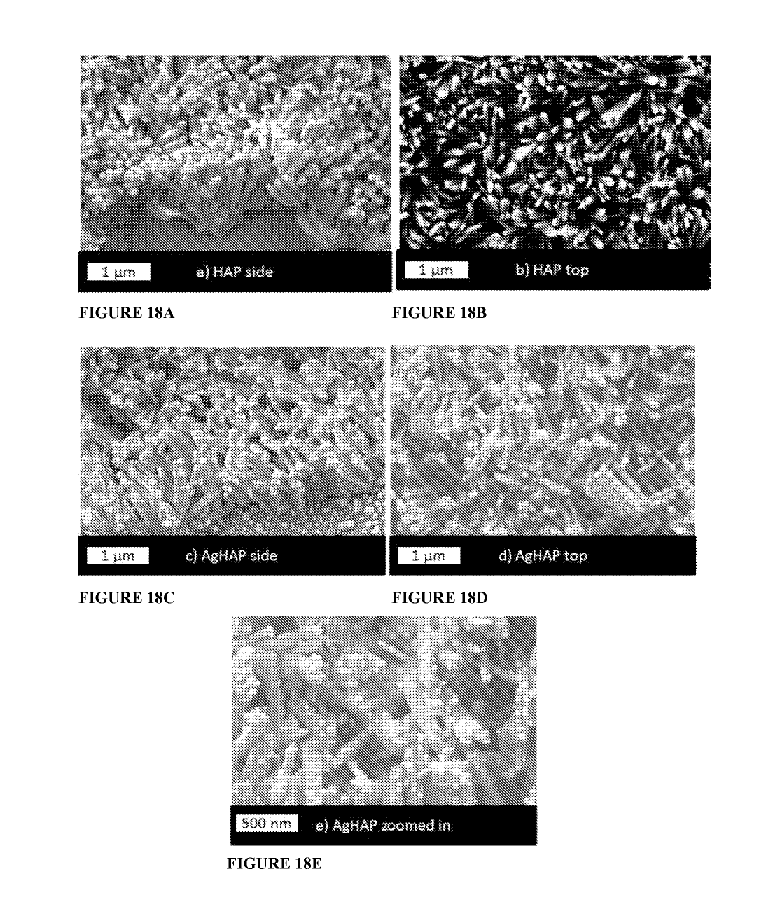

FIG. 21 compares HA, AgHA, and a piece of titanium after being immersed in simulated body fluid (SBF) for 24 hours. This is a simple, acellular bioactivity test used to evaluate a coating's ability to stimulate osseointegration. Both the HA and AgHA samples clearly have a thick new apatite layer formed on and in between the crystals. The thickness of the new apatite layer is approximately the same for both samples, showing that the silver does not impede the apatite-forming ability of the HA coating. EDX results in Table 3 verify that the composition is indeed that of apatite. Titanium, on the other hand, clearly shows its lack of bioactivity. Most of the titanium surface was exposed except for a few small NaCl and CaCl₂ deposits.

TABLE 3
EDX of new apatite layer after immersion
in SBF for HA, AgHA, and Ti.

| Elements (At %) | HAP SBF | AgHAP SBF | Ti SBF |
|---|---|---|---|
| Ca | 20.40 ± 1.09 | 20.09 ± 3.07 | 10.02 ± 3.27 |
| P | 15.49 ± 0.75 | 14.86 ± 1.42 | 3.90 ± 0.90 |
| O | 58.48 ± 1.28 | 59.37 ± 4.06 | 46.79 ± 14.31 |
| Ag | — | 0.89 ± 0.37 | — |
| Na | 2.26 ± 1.09 | 2.19 ± 0.51 | 10.00 ± 3.01 |
| Cl | 1.88 ± 1.01 | 1.23 ± 0.37 | 29.29 ± 11.04 |
| Mg | 1.48 ± 0.10 | 1.38 ± 0.12 | — |

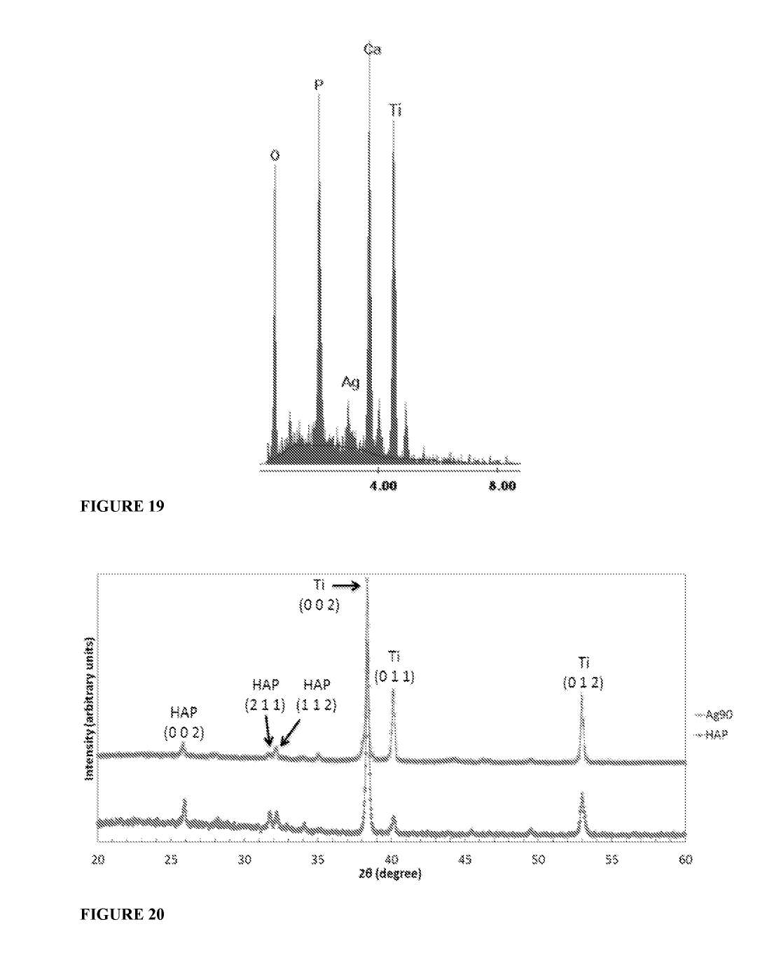

FIG. 22 shows the growth profile of S. aureus bacteria and when it is exposed to the HA and AgHA samples. There is a clear distinction between the two samples, showing the ability of the silver ions released from the AgHA samples to retard bacteria growth. Based on the SBF results, more Ag can potentially be deposited onto the HA surface in order to further reduce bacteria growth, while also not hindering new apatite formation. Future tests should be done to determine the cytotoxicity of increased amounts of Ag nanoparticles.

Conclusions. A simple method to electrochemically reduce Ag nanoparticles onto bioactive HA was developed and characterized. The electrochemical reduction is a fast process that uniformly deposits Ag nanoparticles over the entire HA coating, while not requiring any harsh reducing agents. The AgHA coating has comparable bioactivity to the HA coating according to SBF results, but also has antimicrobial properties.

### Example 4

This example describes preparation and characterization of polarized hydroxyapatite films.

Described is a method to create composite coatings that consist of polarized hydroxyapatite (HA) crystals and metal nanoparticles. The method involves two steps:

1) Electrochemical or electrochemical/hydrothermal synthesis of hydroxyapatite on a metallic substrate: In this method, the metallic substrate acts as the electrode during the electrochemical growth of HA. Optionally, a second hydrothermal synthesis step can be applied to grow additional HA onto the electrochemically deposited material. Thin and uniform coatings of HA are produce consisting of crystals that are preferentially oriented with the crystallographic c-axis normal to the substrate. As a result of the preferred crystal orientation, the coating displays an enhancement in proton conductivity and can retain strong electrical polarization. In the proof of concept data shown below, the HA coating is deposited on pure titanium, but this method can be applied to a variety of metallic substrates including alloys and pure metals.
2) Electrochemical reduction of metal ions to form metal nanoparticles on the coating: The HA coated metal substrate in submerged in a metal salt solution and acts as the electrode for the electrochemical reduction of metal ions to form metal particles. In this example, silver particles are produced, but the method can be extended to electrochemical reduction of a variety of metals in order to form nanoparticles on the HA coating. The size and surface coverage of metal can be controlled by the salt concentration, time, temperature and electrical current used.

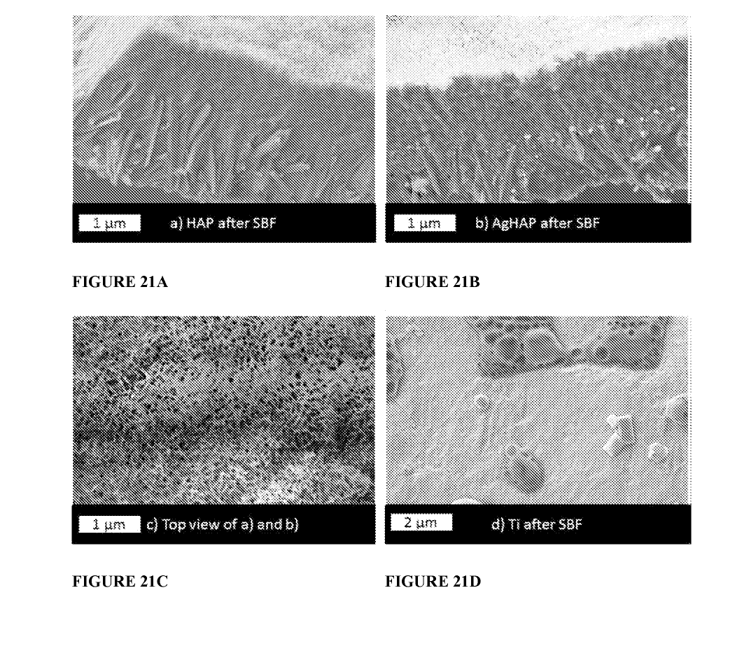

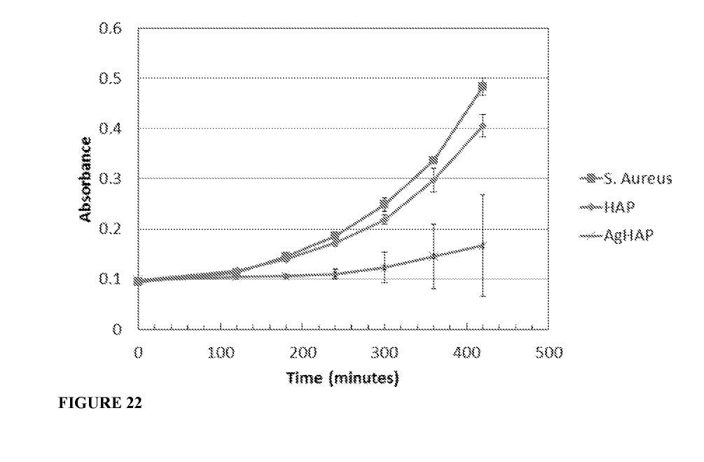

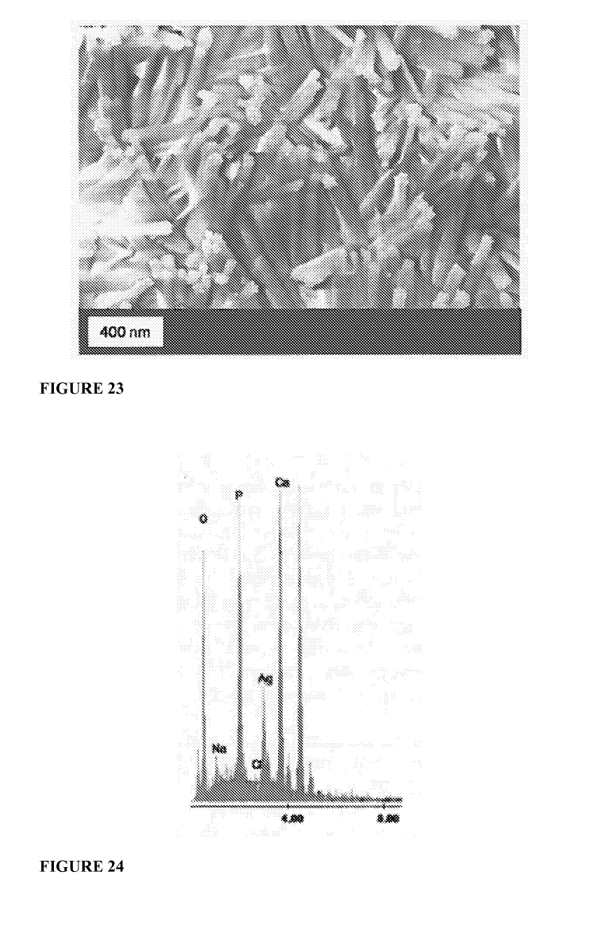

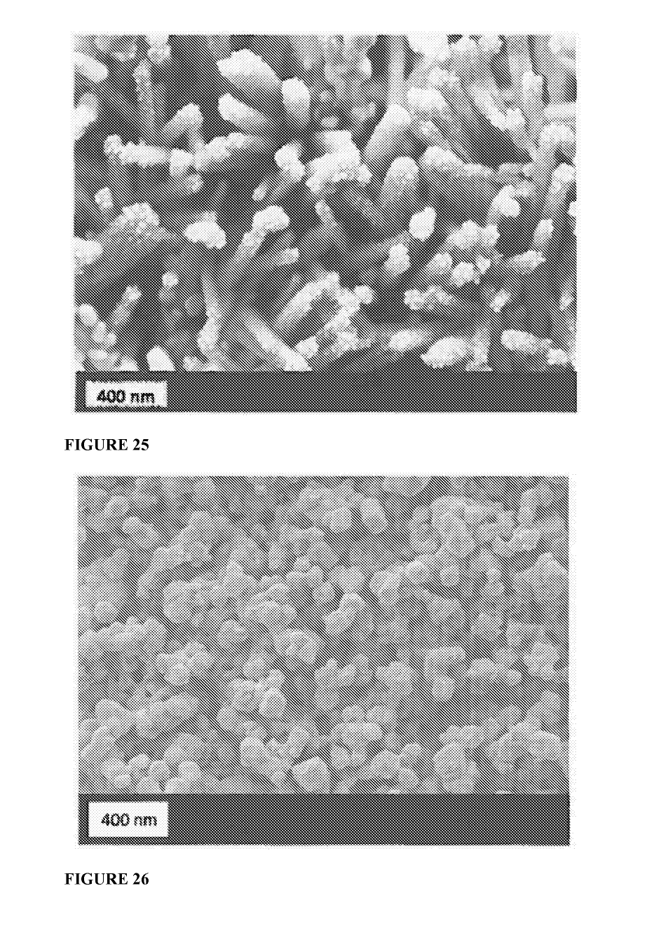

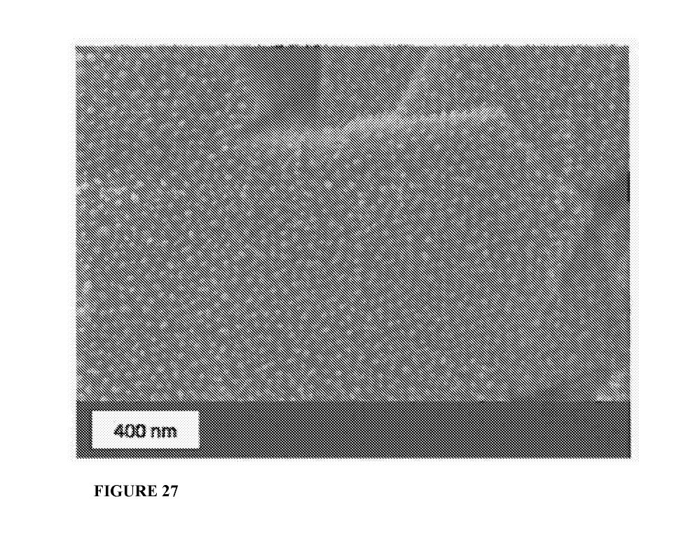

FIG. 23 shows silver nanoparticles preferentially deposited on the tips of the rod shaped HA crystals. Since the deposition time was short and concentration of silver nitrate was low, the silver was primarily deposited as nanometer scale particles at the tips of the HA crystals. FIG. 24 shows results of energy dispersive X-ray spectroscopy that confirms the presence of silver (Ag) on the tips of the HA crystals. FIG. 25 demonstrates that by increasing the deposition time, silver nanoparticles cover the entire exposed surface of the HA crystals. FIG. 26 shows the result of increasing both the concentration of silver nitrate and the deposition time. A dense film of silver particles completely covers the underlying HA crystals. Finally, FIG. 27 shows the deposition of silver nanoparticles on the surface of large HA crystals that were synthesized using a reported electrochemical-hydrothermal synthesis method. The composite coatings shown in the proof of concept may find utility in biomedical applications, particularly in dental and orthopedic implants. Silver provides antimicrobial properties, while the polarized HA stimulates bone growth. The composite coatings therefore have the potential to reduce incidents of infection following implant surgery while simultaneously reducing the patient recovery time. The metal nanoparticles are strongly adherent to the polarized HA coating through electrostatic attraction. The method may also be used to deposit other metal nanoparticles that are catalytically active.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1) A polarized hydroxyapatite film disposed on a substrate, the film comprising a plurality of rod-shaped crystals having a crystallographic c-axis substantially normal to the substrate and the film having a residual electrostatic charge of at least 5 mC/cm².

2) The polarized hydroxyapatite film of claim 1, wherein the film has a gradient composition.

3) The polarized hydroxyapatite film of claim 1, wherein the film has a thickness of 100 nm to 50 microns.

4) The polarized hydroxyapatite film of claim 1, wherein the substrate is a metal substrate, a metal alloy substrate, or graphite substrate.

5) The polarized hydroxyapatite film of claim 1, wherein the substrate is an artificial hip, artificial spine, artificial neck, artificial elbow, artificial femur, artificial knee, artificial shoulder, artificial arm, artificial finger, screw, pin, rod, or a portion thereof.

6) The polarized hydroxyapatite film of claim 1, further comprising a plurality of metal nanoparticles disposed on at least a portion of a surface of the film.

7) A method of making a film disposed on substrate, the film comprising polarized hydroxyapatite, comprising the steps of:
a) heating a precursor solution comprising a calcium precursor (e.g., calcium chloride), a phosphate precursor (e.g., potassium phosphate), and, optionally, sodium chloride buffered to a pH of 3 to 11, to a temperature of 50° C. to 100° C.; and
b) applying an electric current at a current density of 1 to 300 mA/cm² to the precursor solution after reaching the desired reaction temperature a);
such that the film comprising polarized hydroxyapatite comprising a plurality of hydroxyapatite crystals having the crystallographic c-axis of the hydroxyapatite is substantially normal to the substrate, the film having a residual electrostatic charge of at least 5 mC/cm², is formed.

8) The method of claim 7, further comprising contacting the film from b) with a metal salt solution such that metal nanoparticles are formed and disposed on at least a portion of the surface of the film.

9) The method of claim 7, wherein the electric current is applied to the precursor solution for 0.1 minutes to 30 minutes.

10) The method of claim 7, further comprising heating the film from b).

11) The method of claim 10, wherein an electric field of greater than 1 kV/cm to 100 kV/cm is applied to the film during at least a portion of the heating.

12) The method of claim 7, further comprising:
c) depositing a plurality of hydroxyapatite crystals by hydrothermal deposition on the film from b); and
d) optionally, doping the hydroxyapatite crystals during deposition by hydrothermal crystallization with cations, anions, or a combination thereof.

13) The method of claim 9, further comprising contacting the film from b), c), or d) with a metal salt solution under electrochemical reduction conditions such that metal nanoparticles are formed and disposed on at least a portion of the surface of the film.

14) The method of claim 13, wherein an electric field of 1 kV/cm to 100 kV/cm is applied to the film during at least a portion of the heating.

15) A device comprising the hydroxyapatite film of claim 1.

16) The device of claim 15, wherein the device is a medical implant, electret, or filter.

17) The device of claim 16, wherein the medical implant is an artificial hip, artificial spine, artificial neck, artificial elbow, artificial femur, artificial knee, artificial shoulder, artificial arm, artificial finger, screw, pin, rod, or a portion thereof.

18) A method of growing hydroxyapatite using a film of claim 1 comprising exposing the film of claim 1 to body fluid or simulated body fluid such that additional hydroxyapatite is deposited on the film of claim 1.

19) The method of growing hydroxyapatite of claim 18, wherein the method is carried out in vitro or in vivo.
